(19) United States
(12) Patent Application Publication
(10) Pub. No.: US 2021/0169233 A1
(43) Pub. Date: Jun. 10, 2021

(54) SLEEP CONTROL AND MANAGEMENT ACROSS MULTIPLE PLATFORM SLEEP AND BED ENVIRONMENTS

(71) Applicants: Ely TSERN, Los Altos, CA (US); Mark HANDEL, Pittsburgh, PA (US); John TOMPANE, Los Altos, CA (US)

(72) Inventors: Ely TSERN, Los Altos, CA (US); Mark HANDEL, Pittsburgh, PA (US); John TOMPANE, Los Altos, CA (US)

(21) Appl. No.: 17/085,724

(22) Filed: Oct. 30, 2020

Related U.S. Application Data
(60) Provisional application No. 62/929,689, filed on Nov. 1, 2019.

(51) Int. Cl. A47C 31/00; A47C 27/08; A47C 21/04; A47C 27/10; A61B 5/00; A61M 21/02

(52) U.S. Cl. CPC A47C 31/008; A47C 27/083; A47C 21/044; A47C 21/048; A61M 21/02; A47C 27/10; A61B 5/4809; A61B 5/0022; A47C 31/005

(57) ABSTRACT

Beds may include environmental control components, which may be temperature control components and pressure adjustment components in some embodiments. The environmental control components may be configured, for example by a controller, to provide different sleep environments based on identity of sleepers using or expected to use the bed. The controller may receive information as to how to configure the components based on one or more sleep profiles for sleepers expected to use the bed. The sleep profiles may be received from a server, or received from a smartphone of a sleeper. The sleep profiles may be deleted from the bed after use.

### CROSS REFERENCE TO RELATED APPLICATION

This application claims the benefit of the filing date of U.S. Provisional Patent Application No. 62/929,689, filed on Nov. 1, 2019, the disclosure of which is incorporated by reference herein.

### BACKGROUND OF THE INVENTION

The present invention relates generally to sleep environments, and more particularly to configuration of a potential multi-user sleep environment and multiple platform sleep environments.

Sleep is generally a universal need for people. Sleep provides many physiological benefits, and a sound night's sleep is often desired by many. Unfortunately, some may not obtain a sound night's sleep, even when sufficient time and preparation for sleep is available.

Different people may desire different sleep settings for a sound night's sleep. Although most people may, within broad parameters, desire a similar sleep setting—e.g., being able to lie down in a reasonably temperate environment—different people may prefer a host of different environments at the detailed level. For example, different people may have different sleep postures, have slightly different circadian rhythms, or a host of other characteristics that imply that somewhat different sleep setting may be preferable for different people.

Arranging an appropriate sleep setting for a particular individual may be difficult, therefore. The difficulty may be compounded by beds that provide a sleep setting for multiple people simultaneously. Usage of such beds may vary based on when the users may go to bed, based on who those users are, and even if usage of the bed by multiple persons varies on a frequent or infrequent basis.

Moreover, a person (or persons) may use multiple different beds on different occasions. The beds may be in different locations. For example, the beds may be in different rooms of a single dwelling, or spread across multiple dwellings. The beds may even include beds used only temporarily, for example beds in hotels or the like. Appropriately arranging sleep settings in such circumstances may be difficult.

### BRIEF SUMMARY OF THE INVENTION

Some aspects of some embodiments determine a number of sleepers in a bed; determines if the sleeper(s) in the bed may be identified; and conditions a sleep environment of the bed based on the number of sleepers and identities of the sleepers. Some embodiments further determine if there is an expectation that the number of sleepers in the bed is expected to change, and takes the expectation into account in conditioning the sleep environment of the bed. In some embodiments user sleep environment profiles for conditioning the sleep environment of the bed are available for identified sleepers, and such embodiments may utilize the user sleep environment profiles in conditioning the sleep environment of the bed. In some embodiments a default user sleep environment profile may be used in conditioning the sleep environment of the bed in the event of an unidentified sleeper.

Some aspects of some embodiments determine an identity of a sleeper on a bed by comparing information from sensors with information relating to users of the bed. In some embodiments the information from sensors comprises information from pressure sensors. In some embodiments the information from pressure sensors is information from an array of pressure sensors. In some embodiments the information from pressure sensors provides a pressure map. In some embodiments the information from pressure sensors provides an indication of weight of the sleeper. In some embodiments the information from pressure sensors provides an indication of a side of the bed the user entered and/or is upon. In some embodiments the information from sensors comprises information from biometric sensors. In some embodiments information regarding time of day at which the sleeper entered the bed is used, along with information relating to times of day the users have or are expected to enter the bed.

In some embodiments conditioning the sleep environment of the bed comprises controlling a temperature of a sleep surface of the bed and/or controlling firmness of the sleep surface of the bed.

Some embodiments provide a bed system, comprising: a sleep surface; sensors configured to sense sleepers on the sleep surface; components for conditioning a sleep environment of the sleep surface; and a controller configured to receive information from the sensors and to provide commands to the components for conditioning the sleep environment, the controller configured determine a number of sleepers on the sleep surface and to provide at least some different commands to the components based on at least a number of sleepers on the sleep surface.

In some embodiments the controller is further configured to determine identities of sleepers on the sleep surface based on information from the sensors and predetermined information regarding at least some of the sleepers. In some embodiments the sensors comprise pressure sensors. In some embodiments the pressure sensors provide information of a pressure map for the sleep surface to the controller. In some embodiments the pressure sensors provide an indication of weight of sleepers on the sleep surface to the controller. In some embodiments the controller is configured to ignore the indication of weight of a one of the sleepers if the weight is sufficiently below a predetermined weight. In some embodiments the pressure sensors provide information of a side of the sleep surface a sleeper enters. In some embodiments the predetermined information regarding at least some of the sleepers comprises information relating to times of day the sleepers are expected to enter the bed. In some embodiments the information relating to times of day the sleepers are expected to enter the bed comprises information of times of day the sleepers previously entered the bed. In some embodiments the predetermined information regarding at least some of the sleepers comprises, for each sleeper, at least one of a side of the sleep surface expected to be entered by the sleeper, a weight of the sleeper, an expected sleep posture of the sleeper, and a time of day the sleeper is expected to enter the bed. In some embodiments the components for conditioning the sleep environment of the sleep surface comprise components for controlling a temperature of the sleep surface and components for controlling firmness of the sleep surface. In some embodiments the controller is configured to provide commands to the components for conditioning the sleep environment of the sleep surface using sleeper environment profiles for each of the sleepers. In some embodiments the components for conditioning the sleep environment of the sleep surface comprise components for separately controlling a temperatures of a right side and a left side of the sleep surface and components for controlling separately controlling firmness of the right side and the left side of the sleep surface. In some embodiments the controller is further configured to determine a side of the sleep surface for each sleeper on the sleep surface. In some embodiments the controller is configured to provide commands to the components for conditioning the sleep environment of the sleep surface for each side of the sleep surface using the sleeper environment profiles for the sleeper on that side of the sleep surface. In some embodiments the sleeper environment profiles include a default profile for use with an unidentified sleeper. In some embodiments the controller is further configured to determine if there is an expected number and identities of sleepers on the sleep surface. In some embodiments the controller is configured to provide commands to the components for conditioning the sleep environment of the sleep surface for both sides of the sleep surface using a single sleep environment profile if the expected number of sleepers on the sleep surface is one sleeper. In some embodiments the controller is configured to utilize a double optimized sleeper environment profile if the pressure sensors indicate two people sleeping together. In some embodiments the controller is configured to determine an expected number of sleepers on the sleep surface based on information of historical use of the bed. In some embodiments the controller is configured to not condition the sleep environment if the expected number of sleepers is zero. In some embodiments the controller is configured to condition the bed for a sleeper prior to the sleeper entering the bed if the expected number of sleepers is non-zero. In some embodiments the controller is configured to condition the bed for an identified expected sleeper prior to the identified expected sleeper entering the bed using a sleep environment profile for the identified expected sleeper. In some embodiments the controller is configured to determine an expected number of sleepers on the sleep surface based on calendar information of a smartphone of a historical sleeper for the bed. In some embodiments the controller is configured to determine an expected number of sleepers on the sleep surface based on location information of a smartphone of a historical sleeper for the bed.

Some embodiments provide a method of conditioning a bed for sleeping, comprising: determining, by a controller, a number of sleepers in a bed; determining identities, by the controller, of the sleepers in the bed; and conditioning a sleep environment of the bed based on the number of sleepers and identities of the sleepers.

In some embodiments the bed provides a sleep surface for two sleepers, and temperature and firmness of the sleep surface for a right side of the sleep surface and a left side of the sleep surface are separately controllable. In some embodiments the sleep environment of the bed is conditioned based on a sleep environment profile associated with each of the sleepers. In some embodiments an identity of the sleeper may be an unknown sleeper, and a default sleep environment profile is associated with the unknown sleeper. Some embodiments further comprise determining whether there is an expectation that the number of sleepers in the bed will change, and conditioning both the right and left sides of the bed based on the sleep environment profile associated with a sleeper in the bed in response to determining that there is no expectation that the number of sleepers in the bed will change, and conditioning a side of the bed of the sleeper in the bed with the sleep environment profile associated with that sleeper and conditioning another side of the bed without the sleeper with the sleep environment profile associated with a sleeper expected to enter the bed. In some embodiments the controller determines a number of sleepers in the bed based on information from pressure sensors of the bed. In some embodiments the controller determines identities of the sleepers in the bed based on information from the pressure sensors in the bed. In some embodiments the controller utilizes historical usage of the bed in determining identities of the sleepers. In some embodiments the controller utilizes historical usage of the bed in determining whether there is an expectation that the number of sleepers in the bed will change. Some embodiments further comprise determining whether there is no expectation that the number of sleepers in the bed will change, and, in response to determining that there is no expectation that the number of the sleepers in the bed will change, conditioning both the right and left sides of the bed based on the sleep environment profile associated with a sleeper in the bed. In some embodiments the controller determines identities of sleepers in the bed based on information provided by a smartphone associated with a sleeper. In some embodiments the sleep environment of the bed is conditioned based on a sleep environment profile provided to the bed by a server. In some embodiments the sleep environment profile is associated with a sleeper, and the sleep environment profile is provided to the bed in response to a request from a smartphone associated with the sleeper. Some embodiments further comprise deleting the sleep environment profile provided to the bed in response to a request received from the smartphone associated with the sleeper.

Some embodiments provide a method for providing control of a sleep environment across multiple sleep platforms.

In some embodiments the method comprises: receiving at least one sleep profile from a first bed configured to condition a sleep environment based on the at least one sleep profile; transporting the at least one sleep profile to a second bed configured to condition a sleep environment based one or more sleep profiles; and conditioning the sleep environment of the second bed using the at least one sleep profile. In some embodiments the at least one sleep profile is transported to the second bed using a smartphone. In some embodiments the smart phone receives the at least one sleep profile from the first bed by way of wireless communications, and the smart phone transmits the at least one sleep profile to the second bed by way of wireless communications. In some embodiments the at least one sleep profile is transported to the second bed by transmitting the at least one sleep profile over a network from the first bed to the second bed. In some embodiments the at least one sleep profile is stored on a server. In some embodiments the at least one sleep profile is transmitted from the first bed to the server over a network. In some embodiments the at least one sleep profile is transmitted from the server to the second bed over a network. In some embodiments the method further comprises determining that the sleep profile should be deleted from the second bed; and deleting the sleep profile from the second bed in response to determining that the sleep profile should be deleted from the second bed. In some embodiments the determination that the sleep profile should be deleted from the second bed is made based upon a determination that a user associated with the sleep profile has checked out of a hotel. In some embodiments the determination is made based upon an input made into a compute device associated with the second bed.

In some embodiments the method comprises: storing at least one sleep profile, for a user, on a server; providing the at least one sleep profile to a first bed configured to condition a sleep environment based on one or more sleep profiles; conditioning the sleep environment of the first bed using the at least one sleep profile; providing information regarding the conditioned sleep environment of the first bed and sleep status of the user to the server for use in modifying the at least one sleep profile; providing the at least one sleep profile to a second bed configured to condition a sleep environment based on one or more sleep profiles; and conditioning the sleep environment of the second bed using the at least one sleep profile. In some embodiments the method further comprises providing information regarding the conditioned sleep environment of the second bed and sleep status of the user to the server for use in modifying the at least one sleep profile. In some embodiments the at least one sleep profile is provided to the second bed in response to the user being assigned a room containing the second bed.

Some embodiments provide a bed system, comprising: a sleep surface; sensors configured to sense sleepers on the sleep surface; components for conditioning a sleep environment of the sleep surface; a controller, within a frame of a bed of the bed system, configured to receive information from the sensors and to provide commands to the components for conditioning the sleep environment based on a sleep profile; and a compute device, external to the frame of the bed of the bed system, for receiving a request to delete the sleep profile from the bed system and, responsive to receipt of the request, commanding deletion of the sleep profile from the bed system. In some embodiments the compute device comprises a tablet computer. In some embodiments receiving the request to delete the sleep profile from the bed system comprises receiving an input to the tablet computer indicating a request to delete the sleep profile from the bed system. In some embodiments the tablet computer is configured to transmit, to the controller, a command to delete the sleep profile from the bed system. In some embodiments the controller deletes the sleep profile from memory associated with the controller.

These and other aspects of the invention are more fully comprehended upon review of this disclosure.

### BRIEF DESCRIPTION OF THE FIGURES

FIG. 1 is a semi-block diagram of a bed in accordance with aspects of the invention.

FIG. 2 is a diagrammatic expanded perspective view of a bed in accordance with aspects of the invention.

FIG. 3 is a semi-sectional side view of a bed in accordance with aspects of the invention, showing pressure adjustment coil cylinders for adjusting firmness of the sleep surface.

FIG. 4 is a semi-block diagram top view of pressure adjustment coil cylinders and indicating pressure sensor data locations of a bed in accordance with aspects of the invention.

FIG. 5 is a table showing mappings to sleep environment conditioning states, in accordance with aspects of the invention.

FIG. 6 is a flow diagram of a process for configuring a sleep environment, in accordance with aspects of the invention.

FIG. 7 is a flow diagram of a process for determining a configuration for a sleep environment, in accordance with aspects of the invention.

FIG. 8 is a flow diagram of a process for determining a number of sleepers, in accordance with aspects of the invention.

FIG. 9 is a flow diagram of a process for identifying sleepers, in accordance with aspects of the invention.

FIG. 10 is a flow diagram of a process for determining an expectation of later sleeper use, in accordance with aspects of the invention.

FIG. 11 is a semi-block diagram of a bed in a networked environment, in accordance with aspects of the invention.

FIG. 12 is a semi-block diagram of a plurality of beds which may be used by a sleeper, with the beds coupled to a network storing a sleep profile for the user, in accordance with aspects of the invention.

FIG. 13 is a semi-block diagram of a bed coupled to local compute devices, which in turn are coupled to a network in accordance, with aspects of the invention.

FIG. 14 is a flow diagram of a process for configuring a bed, of a plurality of beds, for use by a user, in accordance with aspects of the invention.

FIG. 15 is a flow diagram of a process for storing a sleep profile of a user for transfer to multiple beds, in accordance with aspects of the invention.

FIG. 16 is a flow diagram of a process for obtaining a sleep profile of a user for a bed, of a plurality of beds, in accordance with aspects of the invention.

FIG. 17 is a flow diagram of a process for use in conditioning a bed, of a plurality of beds, for use by a user, in accordance with aspects of the invention.

FIG. 18A is a flow diagram of a process for clearing sleep profile information of a user from a bed, in accordance with aspects of the invention.

FIG. 18B is a flow diagram of a process for determining if a sleep profile on a bed should be cleared, in accordance with aspects of the invention.

FIG. 19 is a flow diagram of a further process for providing a sleep profile to a bed, in accordance with aspects of the invention.

FIG. 20 is a flow diagram of a further process for clearing a sleep profile from a bed, in accordance with aspects of the invention

### DETAILED DESCRIPTION

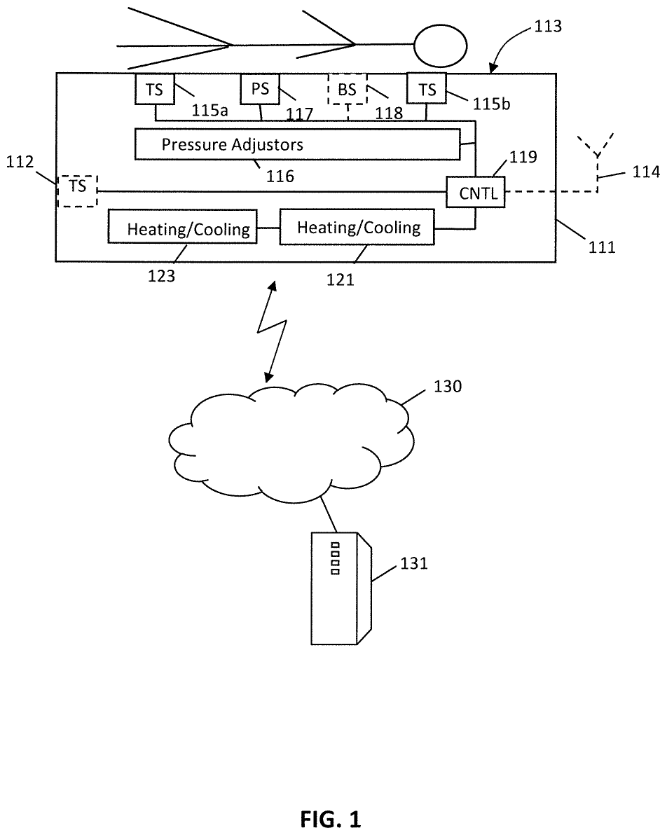

FIG. 1 is a semi-block diagram of a bed 111 in accordance with aspects of the invention. The bed of FIG. 1 includes a sleep surface 113 as an upper surface. In various embodiments, the sleep surface may be a top surface of a mattress, and in some embodiments the mattress, which itself may be comprised of multiple parts (separable or inseparable) may sit on top of a foundation, with the mattress and foundation considered the bed. In various embodiments, however, the bed may include other parts, and in some embodiments the various parts may be combined into one or more separable or non-separable items. The bed of FIG. 1 may be generally rectangular parallelepiped in form, although other forms may instead be used, and in various embodiments may house a variety of components and materials and be comprised of multiple separable components and/or layers. Generally a user, or multiple users depending on the bed, sleeps on the sleep surface.

The bed of FIG. 1 includes components for conditioning a sleep environment. For the example bed of FIG. 1, the components include a heating/cooling component 121 (and optional heating/cooling component 123) and a pressure adjustment component 116. The heating/cooling component allows for adjustment of temperature of the sleep surface of the bed. The pressure adjustment component allows for adjustment of firmness of the sleep surface.

The components for conditioning the sleep environment are generally commanded to do so by a controller 119. In generating commands, the controller may do so using information from sensors, for example temperature sensors 115a,b, pressure sensors 117, and, in some embodiments, biometric sensors 118. The controller also may make use of additional information, for example time-of-day information (for example maintained by the controller), information provided by users by way of user devices, and historical usage and/or sensor information maintained by the controller. In some embodiments the controller may command conditioning of the sleep environment as discussed in U.S. patent application Ser. No. 16/401,108, entitled SLEEP PHASE DEPENDENT TEMPERATURE CONTROL AND LEARNING METHODS TO OPTIMIZE SLEEP QUALITY, filed on May 1, 2019, and/or U.S. patent application Ser. No. 16/401,064 entitled SLEEP PHASE DEPENDENT PRESSURE CONTROL AND LEARNING METHODS TO OPTIMIZE SLEEP QUALITY, filed on May 1, 2019, the disclosures of both of which are incorporated by reference for all purposes herein. As illustrated in FIG. 1, the controller is housed within the bed. In various embodiments the controller can be housed in either the mattress, base or be located externally outside of the bed. In some embodiments the controller comprises one or more processors. In some embodiments the controller is comprised of more than one processor, and the controller may be partitioned and housed in at least two separate physical enclosures, each with at least one processor. In some embodiments the controller is comprised of more than one processor, and the controller may be partitioned and housed in at least two separate physical enclosures, each with at least one processor. In some embodiments the controller is coupled to a network by way of wired or wireless communication circuitry, which may include for example antenna 114. In such embodiments the controller may be coupled (for example by a network 130 which may include the Internet) to a remote server 131, which in some embodiments may perform various of the functions ascribed to the controller herein. In some embodiments the network may include local devices, for example one or more aromatherapy devices and/or air filtrations devices (both not shown in FIG. 1) and/or media devices (not shown in FIG. 1). The aromatherapy device(s) may include various aromas to diffuse, for example as commanded by the controller. In some embodiments, therefore, the aromatherapy device(s) or air filtration devices may also condition the sleep environment. Similarly, the media device(s), which may be audio devices, video devices, and/or audiovisual devices, may provide audio and visual effects, for example sounds, music, and lighting effects, patterns, and displays, for example as commanded by the controller. In some embodiments, therefore, the media device(s) may also condition the sleep environment.

The temperature sensors may be positioned in or adjacent the sleep surface, and provide an indication of a temperature of the sleep surface. In some embodiments, the temperature sensors are worn by the sleeper, provide an indication of a temperature of the sleeper's body or portion of body where the sensor is worn, and can be wired or wirelessly connected to the controller.

The pressure sensors may be located under the sleep surface, and provide an indication of pressure on the sleep surface. Alternatively, the pressure sensors may be located in the controller and connected via air tubes to air chambers underneath the sleep surface to measure the pressure in the air chambers. The biometric sensors may be located in or under the sleep surface, and may provide an indication of heart rate, breathing information, or other biometric information regarding the user on the sleep surface. In some embodiments the biometric sensors may be in an article worn by the user, for example a shirt, with the biometric sensors wirelessly communicating with the controller. In some embodiments the biometric sensors are as discussed or part of an item as discussed in J. Kelly et al., Recent Developments in Home Sleep-Monitoring Devices, ISRN Neurology, vol. 2012, article ID 768794, the disclosure of which is incorporated herein for all purposes.

In various embodiments the controller may condition the sleep environment in accordance with a configuration for the sleep environment determined by the controller. In some embodiments the controller determines the configuration for the sleep environment based on a number of sleepers in the bed, identification of the sleepers in the bed, and an expected later number and identification of sleepers in the bed.

In some embodiments the controller determines a number of sleepers in the bed based on information from the pressure sensors. In some embodiments motion sensors (not shown in FIG. 1) may provide information as to persons in the bed, and the controller may use information from the motion sensors in determining a number of sleepers in the bed.

In some embodiments the controller determines an identity or identities of sleeper(s) in the bed based on comparison of sensor information or other information with predetermined information of candidate sleepers. In some embodiments the controller compares information from the pressure sensors with predetermined information of candidate sleepers. For example, in some embodiments the controller estimates a weight of a person based on the information from the pressure sensors, and compares the estimate of the weight to weights of candidate sleepers, who may have previously provided the controller information regarding their weight. Also for example, in some embodiments the controller generates a pressure map, indicating locus of different pressure on the bed, and compares the pressure map with known pressure maps for candidate sleepers. In some embodiments the controller instead, or in addition, uses information from pressure sensors or motion sensors indicating a side of the bed from which a sleeper entered to identify the sleeper, or information from a microphone (not shown) associated with the bed, or presence of a smartphone in communication, for example via Bluetooth, WiFi or cellular communications, with the controller, or a time of entry into the bed. In some embodiments the controller can communicate and identify a different communication device for each sleeper and can use that identification information to determine which sleeper is close or closest to the bed. In some embodiments the controller with receives location and/or proximity information from the communication device of each sleeper, and uses this information to determine when each sleeper will enter the bed and when to start preparing the sleep environment for each sleeper to be ready when each sleeper enters the bed.

In some embodiments the controller determines an expected later number and identification of sleepers based on historical records for use of the bed. In some embodiments the controller instead or in addition uses calendar information, some of which may be stored in a smartphone or other device and communicated to the controller. For example, in some embodiments the controller maintains a record of indications that a second sleeper historically enters the bed at a certain time of day, which may vary based on the day of the week, or that the second sleeper historically enters the bed on certain days of the week. In such embodiments the controller may also maintain a record of identification of the second sleeper. In addition, in some embodiments the controller may also maintain, or receive, calendar based records of presence of the second sleeper, for example calendar records indicating that the second sleeper is in town or traveling out-of-town.

In some embodiments the heating/cooling component comprises a thermoelectric device, for example a Peltier device. In some embodiments the heating/cooling device comprises a heat pump. In some embodiments, the heating/cooling component may just be a heating component, for example a resistive heater, which in some embodiments may be adjacent or part of the sleep surface. In some embodiments, the heating/cooling component may just be a cooling component, for example an air conditioning device, which in some embodiments may be adjacent or part of the sleep surface. In some embodiments, the heating and/or cooling system includes combinations of heating and/or cooling devices. In the embodiment of FIG. 1 the heating/cooling component is illustrated within the bed, away from the sleep surface. In such embodiments, passageways allowing for thermal transfer between the heating/cooling component and the sleep surface may be provided. For example, in some embodiments, airway passageways are provided between the heating/cooling component and the sleep surface, and some embodiments include other components, for example one or more fans, to assist in conducting heat towards or away from the sleep surface. In some embodiments, where other fluids, for example liquids or fluids that may change between a gaseous and liquid state during use, may be used to control the temperature of the sleep surface, other fluid pathway structures, such as tubes, may be used to move fluids between the active heating and/or cooling component and the sleep surface. Collectively, in some embodiments the temperature control system can include heating and/or heating/cooling components and other fluid (including gaseous fluids) control components, such as fans. The bed may also include a second heating/cooling component 123. The use of a second heating/cooling component may be desirable, for example, in providing differentiation in temperature between different portions of the sleep surface, for example for different sides of the bed used by different users for sleep in a bed normally used by two people. In other embodiments vents or other devices or structures instead or in addition may be used to vary temperature across the sleep surface.

In some embodiments the pressure adjustment component comprises an array of controllable bladders or coils under the sleep surface of the bed. In some embodiments each of the coils is individually adjustable, so as to provide a different level of firmness to the surface of the bed.

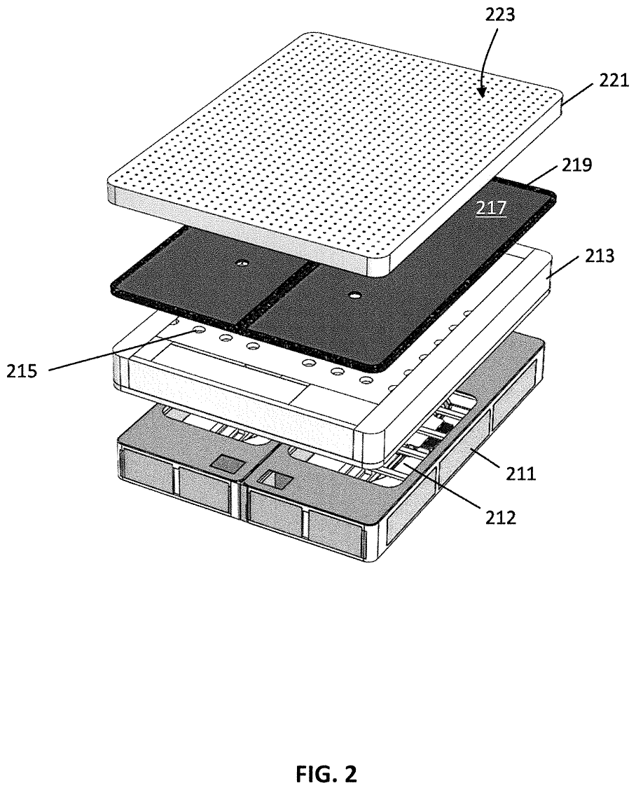

FIG. 2 is a diagrammatic exploded perspective view of a bed in accordance with aspects of the invention. The bed of FIG. 2 may be used as the bed of FIG. 1, in some embodiments.

The bed of FIG. 2 includes a substantially rectangular parallelepiped base 211. The base includes one or more apertures 212 in its upper surface, providing access to a generally open interior of the base. The interior of the base may include, for example, a heating/cooling component, and in some embodiments a controller for controlling the heating/cooling component and other controllable aspects of the bed (although in various embodiments the controller may be elsewhere located in, on, or about the structure of the bed). The apertures in the upper surface of the bed may provide for airflow from the heating/cooling component towards a sleep surface 223 of the bed. In many embodiments legs extend under corners or other portions of the base, for example to allow for the base to be raised off of the floor when in use. In such embodiments the base may also include apertures in a lower surface of the base, for example to provide for further airflow. The base may also include air pumps and associated items for a pressure adjustment component. In some embodiments, and as illustrated in FIG. 2, the base may in fact be two separate bases, positioned to abut one another in a side-by-side configuration.

A mattress of the bed is atop the base. The mattress is comprised of a core 213, with a reticulated foam layer 217 (bounded by a border 219) above the core and a foam pad 221 above the reticulated foam. The core, like the base, is of a generally rectangular parallelepiped form, with generally the same dimensions as the base. The core includes an array of inflatable bladders, which may generally be cylindrical in form and considered coils, as part of the pressure adjustment component. The core also includes apertures extending through the core, from an upper surface of the core to a lower surface of the core. The apertures allow for airflow from the base to the reticulated foam. The reticulated foam diffuses airflow from the core, such that air may diffusely reach the foam pad. Other embodiments may use other materials, such as other types of open structured fibers, that perform similar function of diffusing airflow.

The foam pad, in the embodiment of FIG. 2, also includes apertures through the pad, from an upper surface of the foam pad to a lower surface of the foam pad. The upper surface of the foam pad provides a sleep surface of the bed.

In operation, the heating/cooling component, under command of the controller, causes air of a desired temperature to exit the base and flow, through the core, reticulated foam, and pad, to the sleep surface. The pressure adjustment component, under command of the controller, individually adjusts pressure of the coils, and therefore adjusts firmness of different portions of the sleep surface.

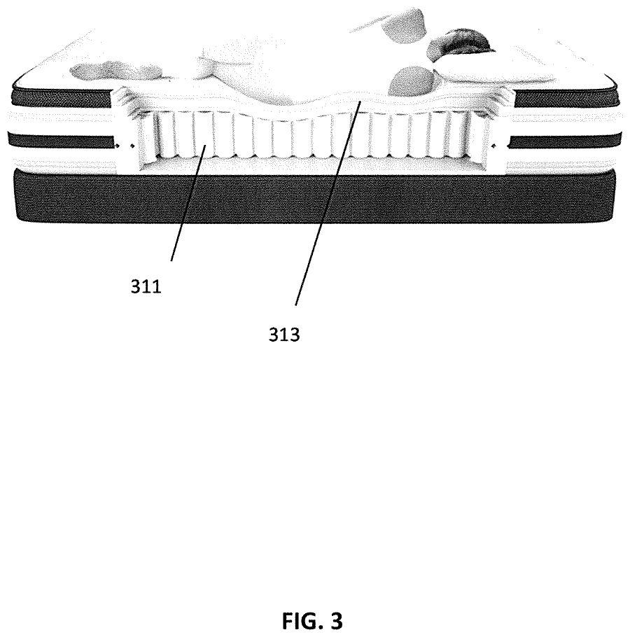

FIG. 3 is a semi-sectional side view of a bed in accordance with aspects of the invention, showing pressure adjustment coil cylinders for adjusting firmness of the sleep surface. The bed of FIG. 3 includes a sensor layer 313 just underneath a sleep surface of the bed. Adjustable coils 311, which may in the form of air bladders, are underneath the sensor layer, and provide adjustable support for a sleeper on the sleep surface.

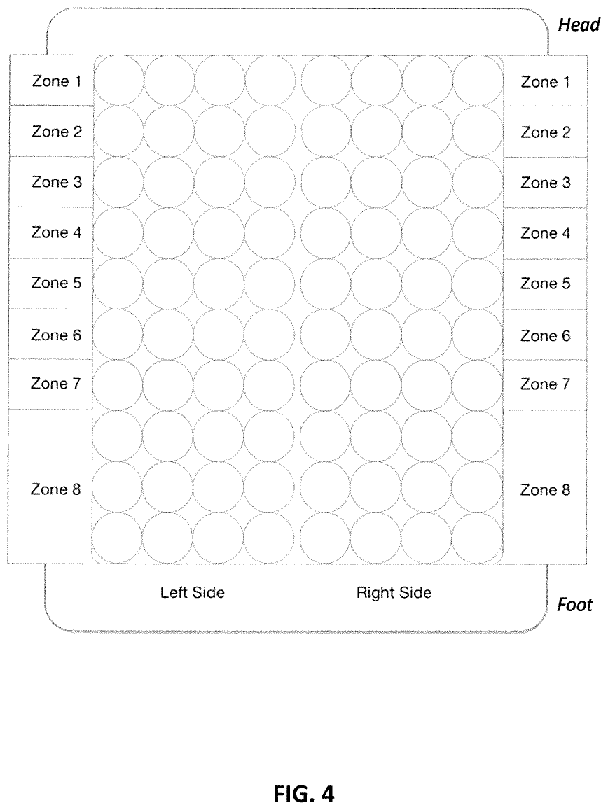

FIG. 4 is a semi-block diagram top view of pressure adjustment coil cylinders and indicating pressure sensor data locations of a bed in accordance with aspects of the invention. The bed includes a left side and a right side. Generally each side is sized to accommodate a sleeper. The bed also includes what may be termed a head of the bed, stretching across a first end of the left and right sides, with a foot of the bed at a second end, opposite the first end. Sleepers will generally position their heads toward the head of the bed, with their feet towards the foot of the bed.

The bed includes an array of pressure adjustment coils or bladders. For the bed of FIG. 4, the array includes 80 bladders, arranged in a 10×8 array. In some embodiments each of the 80 bladders may be individually adjusted. For example, in some embodiments, pressure in each bladder or a group of bladders may be individually regulated, for example as commanded by the controller of FIG. 1.

In some embodiments a pressure fabric or mat or the like may be used to provide pressure indications to a controller. In some embodiments a pressure sensor is associated with each of the bladders. In such embodiments, the controller may receive an indication of pressure on the sleep surface about the location of each of the bladders. In some such embodiments the pressure sensor is positioned in the bed between the bladder and a sleep surface of the bed. In other of some such embodiments, the pressure sensor is associated with an air valve of a bladder or group of bladders.

In some embodiments a pressure sensor is associated with a plurality of bladders. For example, in the embodiment of FIG. 4, a first pressure sensor may be associated with a portion of a row of bladders closest to the head and on the left side of the bed, a second pressure sensor may be associated with a portion of the row of closest to the head and on the right side of the bed, and so on for each row of bladders. Alternatively, some (or all) of the pressure sensors may be associated with bladders of multiple rows. For example, in FIG. 4, a single pressure sensor may be provided for 16 zones, with eight zones on the left side of the bed and eight zones on the right side of the bed, each zone, other than zones closest to the foot of the bed, being for a single row of bladders, with the zones closest to the foot of the bed being for three rows of bladders.

In some embodiments the controller uses the information to determine one, some or all of a side of the bed entered by a sleeper, an estimation of weight of the user (for example based on a summation of pressure indications), a sleep position of the user (for example by a layout indicated by the pressure indications), and a number of sleepers (for example by pressure indications on both the left side and right side of the bed). In some embodiments the controller may also be configured to disregard some pressure indications, for example those for which a determined weight or layout indicates presence something other than a sleeper. For example, in some embodiments the controller may determine that the determined weight and/or layout indicates presence of a cat or dog on the bed, or one or more children on the bed (for example for cases in which the controller has not been provided information that a sleeper may be a child).

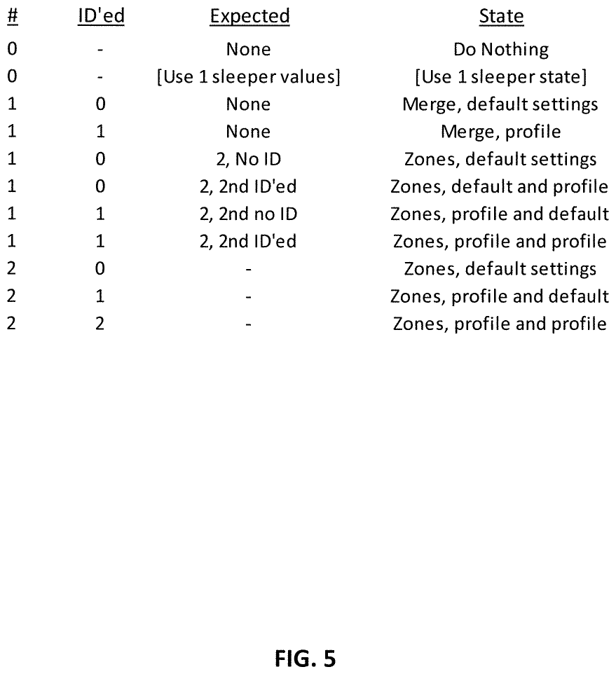

FIG. 5 is a table showing mappings to sleep environment conditioning states in accordance with aspects of the invention. The table includes a first column for number of sleepers in a bed, a second column for the number of the sleepers that are identified, a third column for a number of expected sleepers and whether the expected sleepers are identified, and fourth column for a state to which the bed should be configured. In some embodiments a controller, for example the controller of FIG. 1, determines information for the first three columns based on sensor information and historical and/or calendaring information, with the controller determining a row for selection of the state based on the first three columns.

The number of sleepers in the bed may be determined by the controller based on pressure sensor information, for example. The pressure sensor information may be used to determine whether a person (simply termed a sleeper) or more than one person is on the bed. Although some beds may accommodate more than two sleepers, for the purpose of discussion relating to FIG. 5, it will be assumed that the bed may accommodate up to two sleepers.

In some embodiments the controller identifies the sleeper(s) in the bed based on pre-configured information. For example, in some embodiments a sleeper may provide the controller with some or all of information as to a side of the bed to be entered by the sleeper, a weight of the sleeper, an expected sleep posture of the sleeper, a time of day the sleeper expects to enter the bed, or some other information. In some embodiments the sleeper may provide the controller such information by way of wireless communications from a smartphone device (for example to Bluetooth or Wi-Fi communication circuitry of or associated with the controller). In some embodiments the sleeper may provide the controller such information through an audio interface, for example a microphone, associated with the controller. In some embodiments the sleeper may provide the controller such information by way of a setup procedure for the bed. In some such embodiments the controller may also utilize information from pressure sensors and/or biometric sensors in identifying the sleeper. For example, the pressure sensors may indicate a side of the bed entered by the sleeper, the weight of the sleeper, and/or the sleep posture of the sleeper. Also for example, the biometric sensors may indicate a heart rate of the sleeper or other biological aspects of the sleeper, which also may be compared against information provided to the controller for use in identifying the sleeper.

In some embodiments the controller determines a number of expected sleepers and whether they are identifiable based on historical information or other information provided to the controller. For example, the controller may have been provided information that a second identified sleeper will be joining a first identified sleeper at a certain time of day every day. Alternatively, the controller may determine, for example based on pressure sensor information, that a second unidentified sleeper joins the first identified sleeper every other Tuesday.

Based on the number of sleepers, whether the sleepers are identified, and expected sleepers, the controller selects a state for configuring the bed. For example, if there is 1 sleeper, the sleeper is identified, and no later sleepers are expected, the controller may configure the bed to operate temperature controls for the both the left side and right side the same (e.g. merge the left zone and the right zone) and in accordance to a profile previously determined for the identified individual, and to operate the pressure controls for a single individual, again according to the profile for the identified individual. Alternatively, if there is 1 sleeper, the sleeper is identified, and an unidentified sleeper is expected to join the identified sleeper, the controller may configure the bed to operate the temperature controls and pressure controls for the zone occupied by the identified sleeper in accordance with the profile for that sleeper, and to operate the temperature controls and pressure controls for the other zone according to a default profile. Also for example, if there is 1 unidentified sleeper and some other entity that does not match a weight and/or layout of a sleeper, and no later sleepers are expected, the controller may configure the bed to operate temperature controls for the both the left side and right side the same (e.g. merge the left zone and the right zone) and in accordance to a default profile, and to operate the pressure controls for a single individual, again according to the default profile.

In various embodiments other states may in addition be used. In addition, in some embodiments selection of a state may utilize additional information, for example relative location of sleepers on the bed. The relative location of sleepers on the bed may be indicated, for example, by pressure sensors associated with the bed.

For example, when two sleepers move or start close to sleep together on the left or right side, the controller may, on the double occupied side, change the environment of the double occupied side to a configuration for which the bed environment modified for two people sleeping together, and may use a double occupied profile for doing so. For example, control of the temperature of the sleep surface may be commanded to a lower temperature in some embodiments, for example to account for increased warmth due to the proximity of the sleeper's bodies. In some such embodiments the controller may retain the prior configuration for the unoccupied side, for example in the event the unoccupied side is later re-occupied by the sleeper. Alternatively, in some embodiments the controller may merge both sides into one zone with the entire bed using a profile for couple-optimized configuration environment.

Also for example, when two sleepers move close to sleep together about a middle of the bed, in some embodiments the controller may merge both sides into one zone with the entire bed using the couple-optimized configuration environment. Alternatively, in some embodiments the controller may keep both sides the same as if both sleepers are completely on their side. Further, in some embodiments the controller may determine that two sleepers are in the bed with, for example, a child. In such embodiments the controller may configure environmental control of the bed to, for example merge environmental control of both sides of the bed into one zone, which in some embodiments may be controlled using a profile configuration for a combined couple and child.

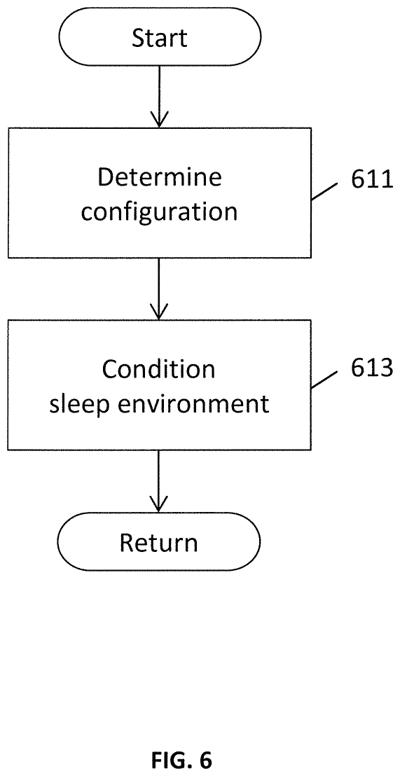

FIG. 6 is a flow diagram of a process for configuring a sleep environment in accordance with aspects of the invention. In some embodiments the process is performed by a bed with a heating and/or cooling component and/or pressure adjustment component. In some embodiments the process is performed by a controller of a bed with a heating and/or cooling component and/or pressure adjustment component. In some embodiments the process is performed by a controller, which may be a processor, for example configured by program instructions.

In block 611 the process determines a configuration for control of a sleep environment of a bed. In some embodiments the configuration for control of the sleep environment is a configuration for control of temperature and/or pressure for a sleep surface of the bed. In some embodiments the configuration for control of the sleep environment additionally includes a configuration for aromas and/or audio and/or video in an area about the bed. In some embodiments the bed includes multiple zones and the configuration for control of the sleep environment provides for merger of control of the multiple zones. In some embodiments the bed includes multiple zones and the configuration for control of the sleep environment provides for separate control of the multiple zones. In some embodiments the configuration for control of the sleep environment configures control of the sleep environment based on one or more sleeper sleep environment profiles. In some embodiments the process determines the configuration for control of the sleep environment based on a number of detected sleepers in the bed, and/or identification of the detected sleepers, and/or an expected number of and/or identification of later sleepers for the bed. In some embodiments the process ignores any entities on the bed for which weight, layout or other information indicates an entity which should not be considered a sleeper (e.g., a cat, a dog, or, in some embodiments, a child). In some embodiments the sleeper sleep environment profiles are profiles for the identified sleepers. In some embodiments the sleeper sleep environment profiles comprise a default profile. In some embodiments the default profile is used in the event a sleeper is not identified.

In block 613 the process conditions the sleep environment. In some embodiments the process commands a temperature for the configured zones of the sleep surface in accordance with one or more sleeper sleep environment profiles (which may be sometimes termed sleep profiles). In some embodiments the process commands a temperature for the configured zones of the sleep surface as discussed in U.S. Provisional Patent Application No. 16/401,108, entitled SLEEP PHASE DEPENDENT TEMPERATURE CONTROL AND LEARNING METHODS TO OPTIMIZE SLEEP QUALITY, and filed on May 1, 2019, the disclosure of which is incorporated by reference herein for all purposes. In some embodiments the process commands firmness for the configured zones of the sleep surface in accordance with one or more sleeper sleep environment profiles. In some embodiments the process commands firmness for the configured zones of the sleep surface as discussed in U.S. Provisional Patent Application No. 16/401,064, entitled SLEEP PHASE DEPENDENT PRESSURE CONTROL AND LEARNING METHODS TO OPTIMIZE SLEEP QUALITY, and filed on May 1, 2019, the disclosure of which is incorporated by reference herein for all purposes. In some embodiments the process commands aroma(s) in accordance with one or more with one or more sleeper sleep environment profiles. In some embodiments the process commands audio and/or video outputs with one or more sleeper sleep environment profiles. In some embodiments the process, at times, may perform a learning function to determine if a measure of sleep quality for the sleeper improves with different temperatures and/or firmness than those indicated by the one or more sleeper sleep environment profiles. For example, in some embodiments the process varies, at times, the commanded temperature and/or firmness to a different temperature and/or firmness than indicated by the one or more sleeper sleep environment profiles, and the process, or another process, may determine if a measure of sleep quality for the sleeper improves with the different temperature and/or firmness. In such embodiments, the process, or another process, may change the temperature and/or firmness indicated by the one or more sleeper sleep environment profiles to indicate use of the different temperature and/or firmness. In some embodiments, the learning and improvement process over multiple nights for a given user can occur across multiple beds, and the improvements that occur across multiple bed platforms are stored in the same user sleep profile. In some embodiments a sleeper may be provided an option, for example by way of a software application executing on a user device in communication with a bed or the server, to disable learning for a particular bed, to disregard learning acquired from a particular bed, or to create a sleep profile specific to a particular bed. For example, in some embodiments different particular beds may be expected to be used only in circumstances expected to significantly vary from a usual routine for a sleeper, for example only for a few nights after the sleeper has traveled across multiple time zones. In such circumstances, a sleep profile specific to such circumstances may or may not have applicability to the sleeper's general sleep preferences.

The process thereafter returns.

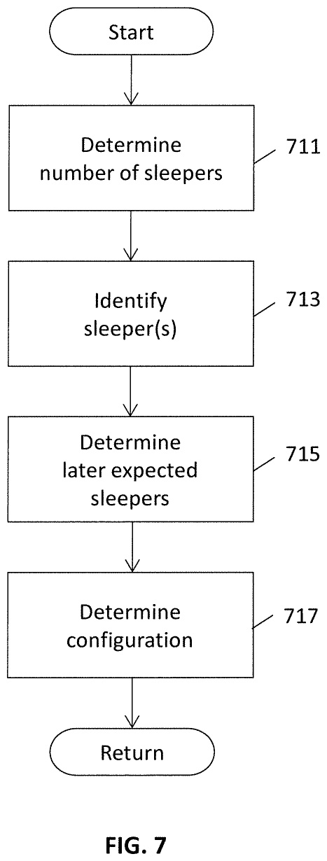

FIG. 7 is a flow diagram of a process for determining a configuration for a sleep environment in accordance with aspects of the invention. In some embodiments the process is performed by a bed with a heating and/or cooling component and/or pressure adjustment component. In some embodiments the process is performed by a controller of a bed with a heating and/or cooling component and/or pressure adjustment component. In some embodiments the process is performed by a controller, which may be a processor, for example configured by program instructions. In some embodiments the process of FIG. 7 is used to perform operations of block 611 of the process of FIG. 6.

In block 711 the process determines a number of sleepers in the bed. In some embodiments the process determines the number of sleepers based on information from pressure sensors of the bed. In some embodiments the pressure sensors provide information of a pressure map for a sleep surface of the bed, and the process determines the number of sleepers based on information of the pressure map. For example in some embodiments sleepers may be expected to exert pressure over particular areas or particular lengths of the surface of the bed. Presence of such pressures may indicate a sleeper, while the lack of such presence of pressures, even if some pressures are indicated (by for example a cat), may indicate a lack of a sleeper. In some embodiments the pressure sensors provide information of an estimation of weight of sleepers in the bed, and the process determines the number of sleepers based on the estimation of weight of sleepers in the bed. In some such embodiments sleepers may be expected to be over a certain weight, and entities weighing less than the certain weight may not be considered sleepers. In some embodiments the pressure sensors provide information as to side(s) of the bed entered by sleepers, and the process determines the number of sleepers based on the number of sides of the bed used for entry by the sleepers.

In block 713 the process identifies sleepers in the bed. In some embodiments the process identifies a sleeper in the bed by comparing information from pressure sensors with information pertaining to one or more sleepers. In some embodiments the information pertaining to the sleeper(s) comprises one, some, or all of a side of the bed normally used for entry to the bed by the sleeper, a weight of the sleeper, and a sleep posture of the sleeper (such as side, back or front, or other postures in between those). In some embodiments the process in addition, or instead, uses an expected time of entry to the bed by the sleeper. In some embodiments the expected time of entry is based on historical records, for example maintained by the controller, of when the sleeper previously entered the bed. In some embodiments the expected time of entry is based on information provided by the user to the controller, for example provided by way of wireless communication between a smartphone of the user and wireless communication circuitry associated with the controller. In some embodiments the expected time of entry is based on location and proximity information provided by the user to the controller, for example provided by way of wireless communication between a smartphone of the user and wireless communication circuitry associated with the controller. In some embodiments the process identifies a sleeper in the bed by comparing information from biometric sensors with information pertaining to one or more sleepers. In some such embodiments, for example, the information pertaining to the sleepers may include a heart rate, a respiration rate, or other information. In some embodiments the process identifies a sleeper in the bed based on an audio input provided by the sleeper, and for example received by a microphone coupled to the controller.

In block 715 the process determines if later sleepers are expected, and the identities of the later sleepers, if determinable. In some embodiments the process determines if later sleepers are expected, and their identities if known, based on historical records of use of the bed, for example maintained by the controller. In some embodiments the process also uses calendar information for sleepers, for example provided to the controller by a smartphone, in determining if later identified sleepers are expected to use the bed.

In block 717 the process determines a configuration for sleep environment for the bed. In some embodiments, for example, the process uses the table of FIG. 5, or information of the table of FIG. 5, in determining a configuration for sleep environment for the bed.

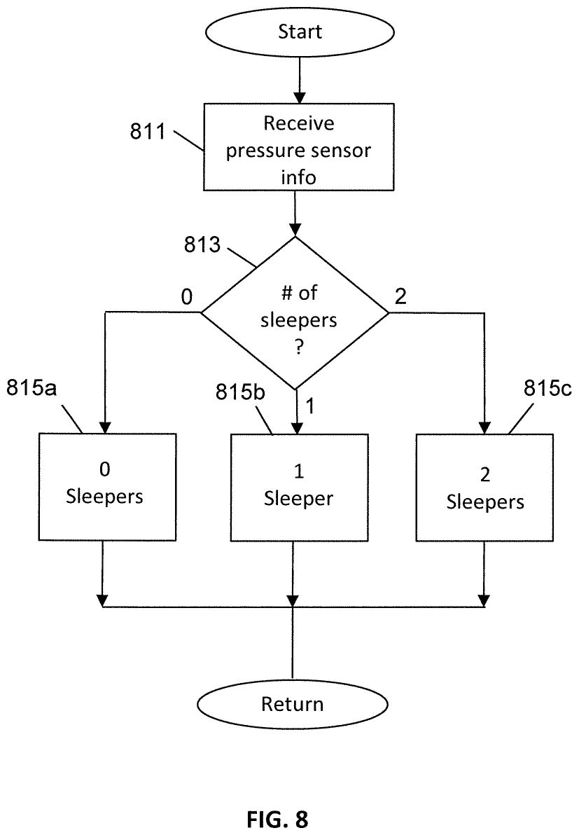

FIG. 8 is a flow diagram of a process for determining a number of sleepers in accordance with aspects of the invention. In some embodiments the process is performed by a bed with a heating and/or cooling component and/or pressure adjustment component. In some embodiments the process is performed by a controller of a bed with a heating and/or cooling component and/or pressure adjustment component. In some embodiments the process is performed by a controller, which may be a processor, for example configured by program instructions. In some embodiments the process of FIG. 8 is used to perform operations of block 711 of the process of FIG. 7.

In block 811 the process receives information from pressure sensors of the bed. The pressure sensors indicate pressure applied to a sleep surface of the bed, for example due to weight of a sleeper being on the bed. In some embodiments the pressure sensors are part of a pressure fabric or mat. In some embodiments the pressure sensors are each associated with a coil or bladder of a pressure adjustment system. In some embodiments the pressure sensors are each associated with a plurality of coils or bladders of a pressure adjustment system.

In block 813 the process determines a number of sleepers on the bed. In some embodiments, and as shown in FIG. 8 for illustrative purposes, the number of sleepers on the bed may range from 0 to 2, inclusive. In some embodiments the process determines a number of sleepers based on information from the pressure sensors indicating a side of the bed from which a sleeper entered the bed; with for example information indicating the bed was not entered resulting in a determination of no sleepers, information indicating the bed was entered from one side by a sleeper resulting in a determination of one sleeper, and information indicating the bed was entered from two sides resulting in a determination of two sleepers. In some embodiments the process determines a number of sleepers based on information from pressure sensors indicating a sleeper on halves of the bed, for example a left half of the bed and a right half of the bed. In such embodiments, information indicating no sleepers results in a determination of no sleepers, information indicating a sleeper on only one half of the bed results in a determination of one sleeper, and information indicating a sleeper on both halves of the bed results in a determination of two sleepers.

For a determination of no sleepers, the process sets a memory location indicating no sleepers in block 815a. For a determination of one sleeper, the process sets a memory location indicating one sleeper in block 815b. For a determination of two sleepers, the process sets a memory location indicating two sleepers in block 815c.

The process thereafter returns.

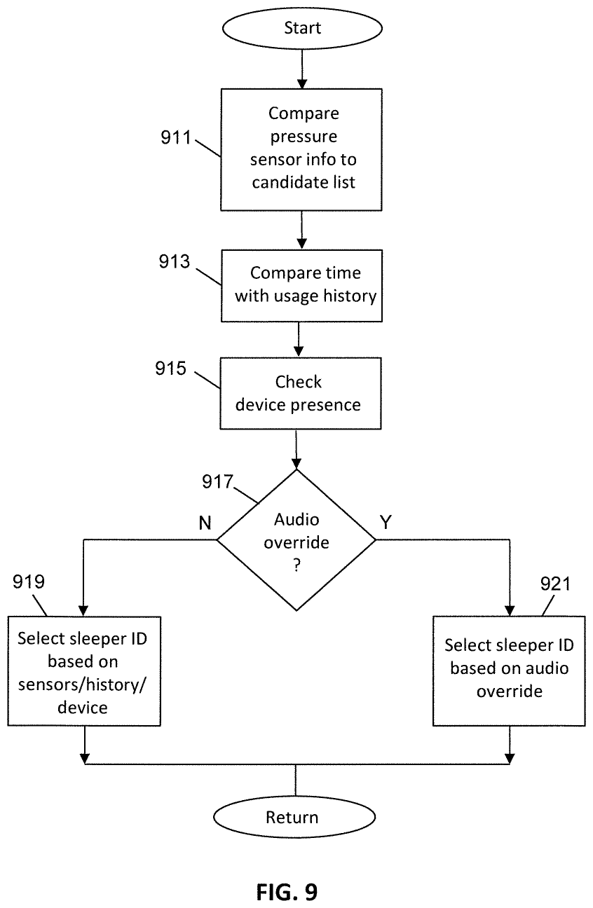

FIG. 9 is a flow diagram of a process for identifying sleepers in accordance with aspects of the invention. In some embodiments the process is performed by a bed with a heating and/or cooling component and/or pressure adjustment component. In some embodiments the process is performed by a controller of a bed with a heating and/or cooling component and/or pressure adjustment component. In some embodiments the process is performed by a controller, which may be a processor, for example configured by program instructions. In some embodiments the process of FIG. 9 is used to perform operations of block 713 of the process of FIG. 7.

In block 911 the process compares information from pressure sensors with expected information for possible candidate sleepers. In some embodiments the information from pressure sensors indicates a half of the bed being slept on by a sleeper, with the half of the bed being either the left half of the bed or the right half of the bed in some embodiments. In such embodiments, the controller, for example, may maintain information as to which half of the bed a particular sleeper sleeps on. In some embodiments each of two sleepers has configured the bed to assign one unique side of the bed to each sleeper, and that configuration information of bed side assignments is stored in the controller and used for determining which sleeper is in the bed for future sessions. In some embodiments the information from the pressure sensors indicates a sleep posture of the sleeper, and the information for possible candidate sleepers includes information as to expected sleep postures of the candidate sleepers.

In block 913 the process compares time of entry to the bed with usage history of the bed by candidate sleepers. For example, in some embodiments the controller maintains a record of time of usage, or a record of time of entry to the bed, by particular sleepers. In block 915 the process checks for presence of communication devices associated with particular sleepers. In some embodiments, the controller has associated wireless communication circuitry, for example Bluetooth or Wi-Fi communication circuitry, and the controller determines if communication with a device associated with particular sleepers is available. In some embodiments the controller can communicate and identify a different communication device for each sleeper and can use that identification information to determine which sleeper is close or closest to the bed. In some embodiments the controller with receives location and/or proximity information from the communication device of each sleeper, and uses this information to determine when each sleeper will enter the bed and when to start preparing the sleep environment for each sleeper to be ready when each sleeper enters the bed. In some embodiments, the criteria to decide whether a sleeper will be expected to sleep in the bed for that night is determined by a predetermined distance between the sleeper's location from their communications device and the bed location at a given time and the amount of remaining time before a scheduled sleep time. In some embodiments, if one sleeper is not expected to sleep in the bed that night based on this criteria, the bed will configure both sides of the bed to the personal configuration of the second sleeper. In some embodiments, if both sleepers are not expected to sleep in the bed that night based on this criteria, the bed will configure both sides of the bed to be turned off for that sleep session.

In block 917 the process determines if an audio input, for example to a microphone associated with the bed, has been received, with the audio input identifying a sleeper. If the audio input has been received, the process goes to block 921, and identifies the sleeper based on the audio input, and thereafter returns. Otherwise the process continues to block 919.

In block 919 the process identifies the sleeper based on the pressure sensor information, the usage history, and device presence, or based on one or some of those. For example, in some embodiments, the comparison of the pressure sensor information with expected information for candidate sleepers may indicate the sleeper is a particular identifiable individual. In some embodiments, the particular identifiable individual may be determined to be the sleeper. In some embodiments the comparison may indicate that the sleeper may be one of several candidate sleepers. In such embodiments, the process may narrow down the candidate sleepers to a particular identifiable sleeper based on, for example, time of entry to the bed and usage history and/or device presence. In some embodiments the process identifies the sleeper as an unidentified sleeper, for example if the process is not able to identify the sleeper as a particular identifiable sleeper.

The process thereafter returns.

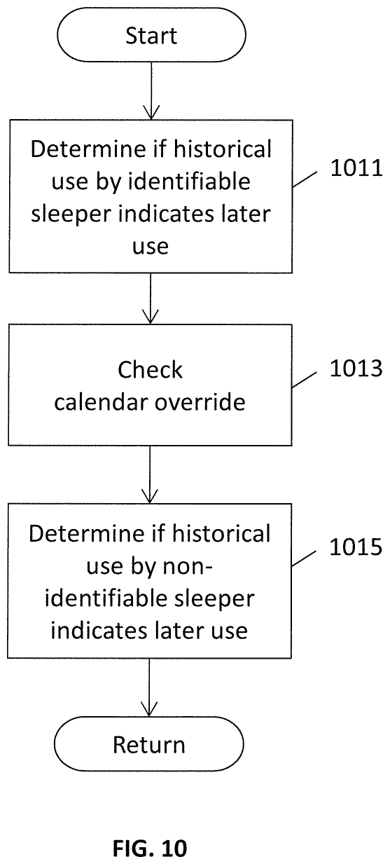

FIG. 10 is a flow diagram of a process for determining an expectation of later sleeper use in accordance with aspects of the invention. In some embodiments the process is performed by a bed with a heating and/or cooling component and/or pressure adjustment component. In some embodiments the process is performed by a controller of a bed with a heating and/or cooling component and/or pressure adjustment component. In some embodiments the process is performed by a controller, which may be a processor, for example configured by program instructions. In some embodiments the process of FIG. 10 is used to perform operations of block 715 of the process of FIG. 7.

In block 1015 the process determines if historical use by an identifiable sleeper indicates later use of the bed. In some embodiments the controller maintains a record of time of entry to the bed by identified sleepers, and the process uses this information to determine if the identifiable sleeper is expected to enter the bed at a later time.

In block 1017 the process determines if historical use by the identifiable sleeper should be ignored based on information from a calendar for the identifiable sleeper. For example, the calendar for the identifiable sleeper may indicate the identifiable sleeper is out of town, with the implication that the identifiable sleeper will not be entering the bed. For example, the GPS location information of the communication of the identifiable sleeper may indicate the identifiable sleeper is out of town, with the implication that the identifiable sleeper will not be entering the bed.

In block 1015 the process determines if historical use by a non-identifiable sleeper indicates later use. For example, the controller may maintain records of entry to the bed of an unidentified sleeper, and such records may indicate, for example, that the unidentified sleeper enters the bed later every Tuesday. In such a circumstance, every Tuesday the controller may determine that an unidentified sleeper is expected to later enter the bed.

The process thereafter returns.

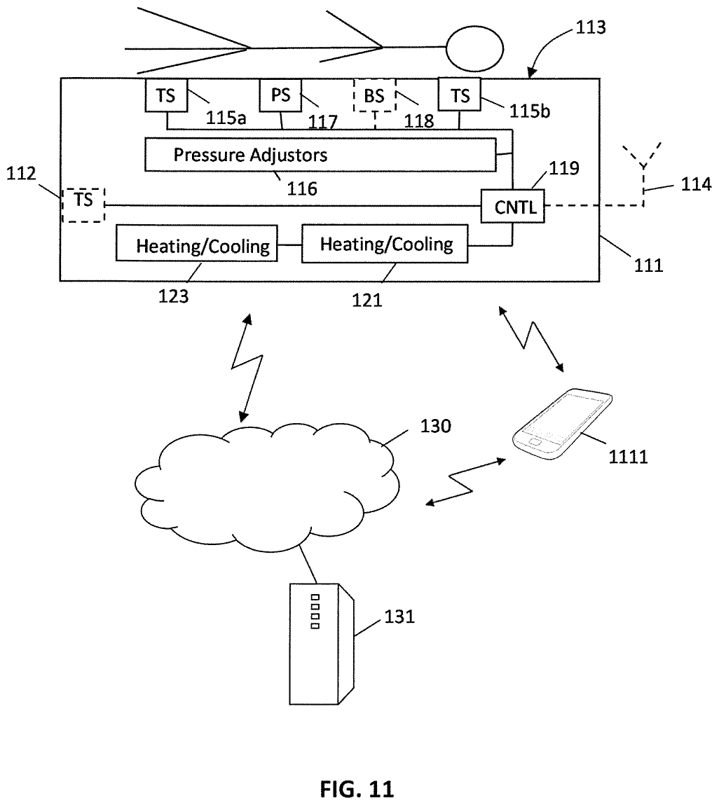

FIG. 11 is a semi-block diagram of a bed in a networked environment in accordance with aspects of the invention. The bed may be the bed of FIG. 1 in various embodiments, and as illustrated in FIG. 11 the bed generally includes the various items of the bed of FIG. 1, including, in some embodiments, aromatherapy, air filtration, and/or media device(s). Accordingly, except as possibly discussed below, reference may be had to the discussion of FIG. 1 for items of the bed.

As discussed with respect to FIG. 1, the controller 119 of the bed of FIG. 11 is coupled to a network by way of wired or wireless communication circuitry, which may include for example antenna 114. In such embodiments the controller may be coupled (for example by a network 130 which may include the Internet) to a remote server 131. As further illustrated in FIG. 11, the controller may also communicate with a sleeper's personal compute device, for example a smartphone 1111 (as illustrated in FIG. 11), a tablet computer, or other device, either directly, for example using Bluetooth communications, through a local network device (not shown in FIG. 11), or through the remote server. In some embodiments the bed, and, in some embodiments, the server and/or the smartphone may be considered to be a system for providing conditioning of a sleep environment in accordance with one or more sleeper profiles.

In some embodiments the smartphone stores information of a sleeper sleep environment profiles for a user associated with the smartphone. The sleeper sleep environment profiles may include information which may be used by the controller in determining commanded sleep surface temperatures and commanded sleep surface firmness for the bed. In such embodiments, the smartphone may provide the sleeper sleep environment profiles to the bed, for use in conditioning a sleep environment for the user. The user may therefore, through use of the user's smartphone, be able to configure a bed, such as the bed of FIGS. 1 and 11, for use by the user.

In some embodiments the smartphone may transmit a request for available configurable beds, with the bed responding to the request, such that the smartphone and bed may thereafter communicate. In some embodiments bed network identification information may be attached to or printed on material on or near the bed, the information being for example an alphanumeric code or QR code or the like. In such embodiments the smartphone may be able to make use of the bed network identification information in arranging communications with the controller of the bed. In some embodiments a password may also be attached to or printed on material on or near the bed, or otherwise made available to the user, for use in arranging communications with the bed. Such may be useful, for example, in avoiding undesired access to the bed.

In some embodiments the remote server may store information of the sleeper sleep environment profiles for the user. In such embodiments the user may, through use of the smartphone or other network device (for example another computer or server coupled to the network), provide the remote server information to identify the bed, and in some embodiments, to allow the remote server to provide information of the sleeper sleep environment profiles to the bed. Accordingly, in some embodiments the remote server may provide information of the sleeper sleep environment to the bed. In some embodiments the bed may provide the smartphone bed identification information, which the smartphone may provide to the remote server. In various embodiments, the smartphone may also provide user identification information to the remote server, along with, in some embodiments, a password or key or the like for use in validating the user ID, or that use of the user ID is an authorized use.

In some embodiments the smartphone may also provide information as to which side of the bed the user will use, and/or whether the user will be the only sleeper in the bed. In some embodiment the user may also be allowed to provide information regarding a second sleeper in the bed, including in some embodiments information of sleeper sleep environment profiles for the second sleeper (if so authorized, for example by way of having authentication permission from the second sleeper). In some embodiments the controller, or the remote server, may request such information from the smartphone, or provide an interface for the smartphone to provide such information.

In some embodiments the smartphone (or other network device, for example a computer or other server coupled to the network) may also provide a request to delete information regarding the user from the bed. In some embodiments the controller may also be configured to delete the information regarding the user after an expected wake time of the user, or at or after the expected wake time and the user is no longer on the bed.

In some embodiments the controller of the bed is also in communication with a networked aromatherapy device, for example in the form of a diffuser. In some embodiments the networked aromatherapy device may include cartridges for diffusing a plurality of different aromas, with a particular cartridge selectable for use by way of a command from the controller. In some embodiments the sleep profile includes indications of aromas to diffuse during different times of the user's sleep experience, for example such as one aroma for before sleep, a second aroma during sleep, and a third aroma for waking up, with the controller commanding use of cartridges providing those aromas.

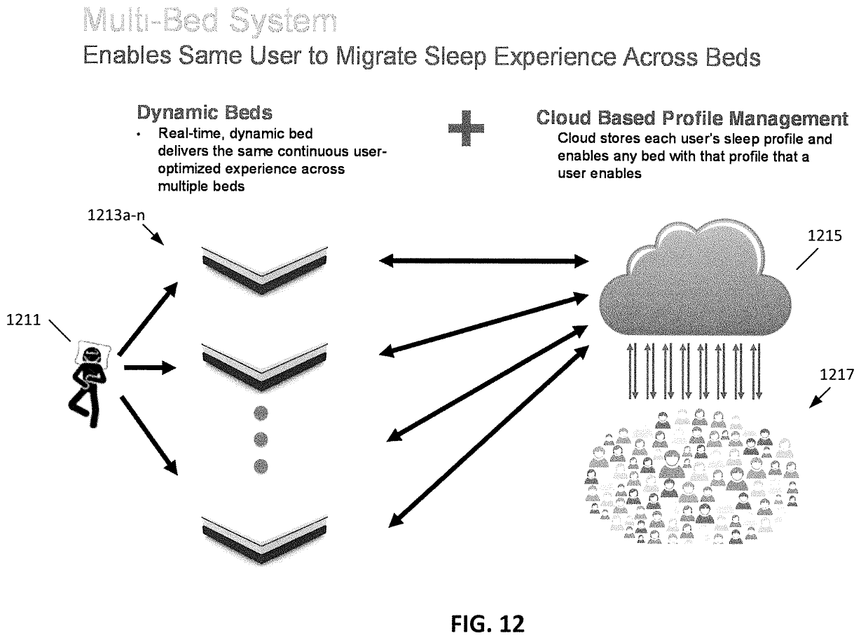

FIG. 12 is a semi-block diagram of a plurality of beds which may be used by a sleeper, with the beds coupled to a network (which may include a server) storing a sleep profile for the user in accordance with aspects of the invention. In FIG. 12, a user 1211 may use any of a plurality of beds 1213a-n. In some embodiments each of the beds may be personal to a particular user, for example beds owned by and in one or more homes of the particular user. In some embodiments one or more of the beds may a bed which a particular user may use, but which others might use as well. For example, in some embodiments, the particular user may own a one of the beds, with the bed in the particular user's home, while another bed is in a hotel or another person's residence, which the particular user may on occasion visit and stay.

Each of the beds may be, for example, a bed discussed with respect to FIG. 11. As discussed with respect to FIG. 11, and by reference FIG. 1, the beds may include components for controlling a sleep environment, including firmness of a sleep surface of the bed and temperature of and about the sleep surface, based on one or more sleep profiles. Each of the beds may receive the sleep profiles from a server accessible to the bed over a network, which may include the Internet in some embodiments. Colloquially, the sleep profiles may be considered to be stored in the “cloud’ 1215. In such embodiments, the sleep profiles may be downloaded from a server in the cloud, and used by the bed in controlling the sleep environment. The bed may, in various embodiments, modify the sleep profiles, for example based on learning operations performed by the bed, and provide the modified sleep profiles to the server. In some embodiments information for use in the learning operations may be provided by the server. In some embodiments, information regarding the sleep environment and user sleep status may be provided by the bed to the server, and the server may modify the sleep profiles. In various embodiments, the sleep profiles for a particular user may therefore be used across multiple beds, and, in various embodiments, modified due to learning operations performed across the multiple beds.

In some embodiments, the beds, or one or some of them, may instead receive the sleep profiles from a personal compute device, for example a smartphone, of a user. In such embodiments the bed may modify sleep profiles as a result of the learning operations, with the bed providing the modified sleep profiles to the smartphone for later use in the same bed, or different ones of the multiple beds. In some such embodiments the server may perform the learning analysis and/or perform the modification of the sleep profiles, for example based on information regarding the sleep environment and user sleep status provided by the bed, with the server providing the sleep profiles to the bed, for provision to the smartphone, or directly to the smartphone over the network. Again, in various embodiments, the sleep profiles for a particular user may therefore be used across multiple beds, and, in various embodiments, modified due to learning operations performed across the multiple beds.

In some embodiments the beds may be discoverable by the smartphones, with for example the smartphone being configurable to transmit interrogation signals, to be responded by controllers of the beds which receive such interrogation signals.

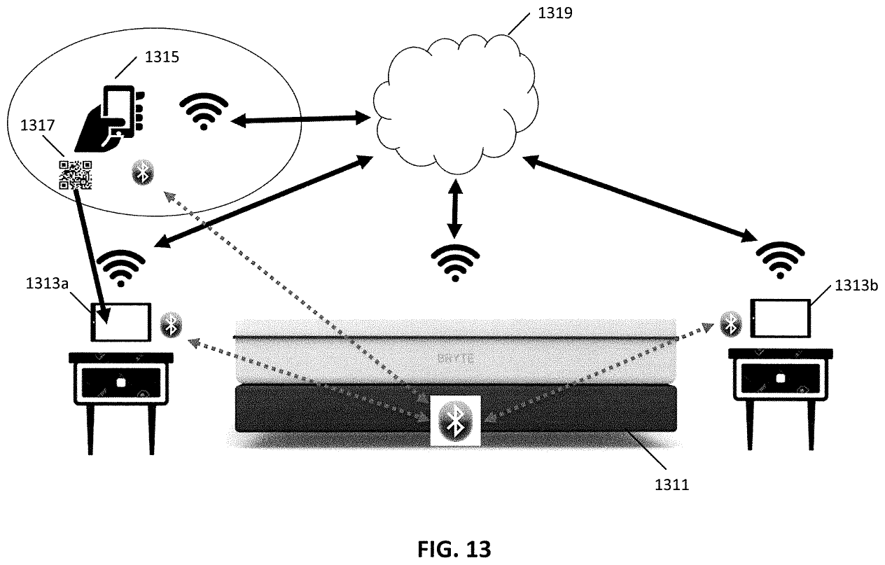

FIG. 13 is a semi-block diagram of a bed 1311 coupled to local compute devices 1313a,b, which in turn are coupled to a network, in accordance with aspects of the invention. The bed may be the bed of FIG. 11, and may be one of the multiple beds of FIG. 12. The bed and/or the compute devices are coupled to a network 1319, which may be the Internet. By way of the network 1319 the bed and/or compute devices may communicate with servers or other computers coupled to the network. In some embodiments the bed and the local compute devices comprise a bed system, with the local compute devices providing a user interface for operation of the bed. Two local compute devices are shown in FIG. 13, for example one for each side of the bed. In some embodiments the bed system may include only a single local compute device. In addition, in FIG. 13 the local compute devices may be considered to be shown as tablet computers, although in various embodiments various other compute devices may be used instead. In some embodiments the bed system may be located, for example, in a residence or hotel guest room, and in some embodiments each of a plurality of guest rooms in the hotel, or all of the guest rooms in some embodiments, may include a bed system.

In some embodiments a user or users may use the compute devices to configure the bed for sleep. In some embodiments the users may enter information into the compute devices, information sufficient to allow the compute devices, or a controller of the bed in some embodiments, to request sleep profiles for the users from a server. In some embodiments the information may be a username or identifier for the user, with in some embodiments use of a password also required. In some embodiments a user may enter information as to a desired sleep profile, or information which may be used to generate a sleep profile for use, or initial use, by a user. For example, a user may enter information regarding the user (e.g. weight, preferred sleep position, etc.) and/or desired user preferences, such as sleep times, awakening times, firmness profile settings, temperature, room environment settings, and/or other information. In some embodiments the user may also enter into the compute device information as to whether the user wishes to create a new sleep profile to be used by the user for different beds at a later time, or merely use a new sleep profile temporarily while making use of the bed. In some embodiments the user may also be provided an option to select a default profile for use while the user makes use of the bed. The compute devices therefore may receive information sufficient to request a user sleep profile from the server, or may receive information sufficient to generate or use a new sleep profile for the user, with the sleep profile possibly being a temporary sleep profile.

In some embodiments a user may make use of a personal compute device, for example a smartphone 1315 of the user, also or instead be used in setting up the bed of FIG. 13 for use with one or more sleep profiles. In some embodiments the compute devices may display a code, for example a numeric code, an alphabet code, an alphanumeric code, or a QR code, which may be entered into or scanned by the smartphone. The code may identify the bed, and an application executing on the smartphone may provide the code to a server, for example to identify the bed to the server so that the server may provide a sleep profile to the bed. In some embodiments the application executing on the smartphone also provides information regarding the user, for example an identifier identifying the user or a sleep profile associated with the user, to the server. The server may therefore provide to the bed the sleep profile associated with the user. In some embodiments the user's personal compute device, for example the user's smartphone, may display a QR code, or other code, with the compute device associated with the bed scanning the QR code or other code and using information of the scanned code to obtain the sleep profile from the server. In some embodiments, when a bed has dedicated compute devices to control the bed, and a user has their own personal compute device in the room, the personal compute device can take over functions otherwise performed by the compute device associated with the bed, in some embodiments in effect taking over control of the bed. In some embodiments, the personal compute device detects the bed in the room and gives the user the option to connect to the bed, or automatically connects to the bed, and the personal compute device becomes a primary control interface for the bed, instead of the compute device associated with the bed, if present.

In some embodiments the compute devices may provide an interface for commanding deletion of sleep profiles from the bed, or deletion of particular sleep profiles (or deletion of all but certain preidentified sleep profiles). In some embodiments the interface may be presented as a button on display screen of the compute devices. In some embodiments, in response to receiving an indication that the button has been pressed, the compute device is configured to provide the controller of the bed a command or request to delete one or more sleep profiles. In some embodiments the command or request is to delete all sleep profiles from the bed. In some embodiments the command or request is to delete all but certain preidentified sleep profiles from the bed. In some embodiments the preidentified sleep profiles may be default sleep profiles. In some embodiments, the compute device first displays an indication of sleep profiles present on the bed, and an option to select sleep profiles for deletion (or for non-deletion).

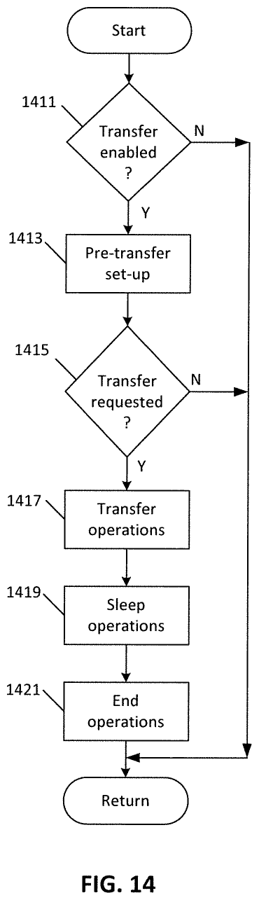

FIG. 14 is a flow diagram of a process for configuring a bed, of a plurality of beds, for use by a user, in accordance with aspects of the invention. In some embodiments the process is performed by a system, for example the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a personal compute device, for example a smartphone, and a controller or controllers of beds with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions.

In block 1411 the process determines if a transfer of a sleep profile from one bed to another bed is allowed. In some embodiments transfer of the sleep profile is allowed if an indication has been received indicating that the sleep profile may be used on multiple beds. In some embodiments the smartphone receives the indication, for example by way of a user input, which may be selection of an option allowing for use of the sleep profile on multiple beds. In some embodiments the smartphone provides the indication to the server. In some embodiments a compute device of a bed system receives the indication, for example by way of a user input, and in some embodiments the compute device may provide the indication to the server.

If a transfer of the sleep profile from one bed to another is allowed, the process continues to block 1413. Otherwise the process returns.

In block 1413 the process performs pre-transfer setup operations. In some embodiments the pre-transfer setup operations comprise storing the sleep profile on the smartphone. In some embodiments the pre-transfer setup operations comprise storing the sleep profile on the server. In some embodiments the bed transmits the sleep profile to the smartphone. In some embodiments the smartphone, in turn, may transmit the sleep profile to the server. In some embodiments the bed transmits the sleep profile to the server. In some embodiments the process may store the sleep profile on the server only if the process has received an indication that the sleep profile may be stored on the server. In some embodiments the indication that the sleep profile may be stored on the server is receipt of a user input, into the smartphone or compute device, indicating that the sleep profile may be stored on the server.

In block 1415, the process determines if a transfer of the sleep profile to another bed is requested. In some embodiments the process determines that a transfer is requested if the process receives a request indicative of a request to download the sleep profile. In some embodiments the request is based on receipt of an input to a smartphone or compute device of a bed system indicating that the sleep profile should be downloaded to another bed. In some embodiments the request includes an identification of a user and/or a sleep profile associated with the user, and the other bed.

If a transfer of the sleep profile from one bed to another is requested, the process continues to block 1417. Otherwise the process returns.

In block 1417 the process transfers the sleep profile to the other bed. In some embodiments the server transmits the sleep profile to the bed. In some embodiments the smartphone transmits the sleep profile to the bed.

In block 1419 the process performs sleep operations. In some embodiments the process conditions a sleep environment of the other bed based on the sleep profile. In some embodiments the sleep environment of the other bed is conditioned using temperature adjustment components of the other bed and/or pressure adjustment components of the other bed, for example as commanded by a controller of the other bed, based on information of the sleep profile. In some embodiments the process also performs learning operations with respect the sleep environment, learning operations which may result in changes to the sleep profile.

In block 1421 the process performs end operations. In some embodiments the process performs end operations upon receipt of an indication that the user will no longer be using the other bed. In some embodiments the receipt of the indication is receipt of an input to the smartphone indicating the user will no longer be using the other bed. In some embodiments the receipt of the indication is receipt of an input to the compute device indicating the user will no longer be using the other bed. In some embodiments the receipt of the indication is receipt of a message from a server that the user will no longer be using the other bed. In some embodiments the end operations include deleting the sleep profile from the other bed. In some embodiments the controller of the other bed commands deletion of the sleep profile from memory of the other bed.

The process thereafter returns.

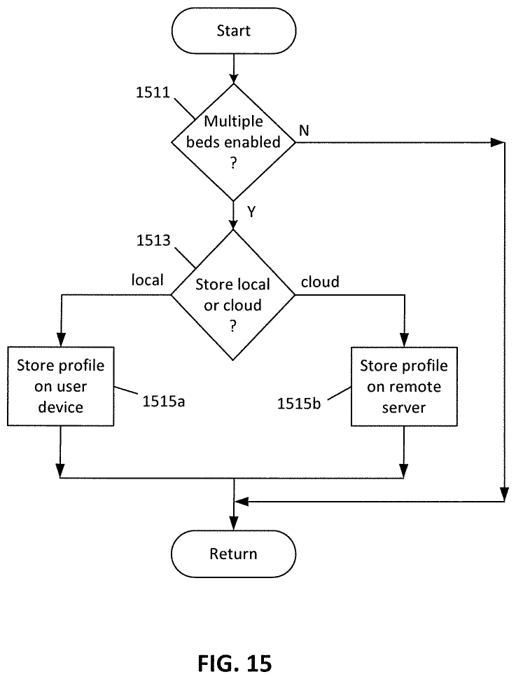

FIG. 15 is a flow diagram of a process for storing a sleep profile of a user for transfer to multiple beds, in accordance with aspects of the invention. In some embodiments the process is performed by a system, for example the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a personal compute device, for example a smartphone, and a controller or controllers of beds with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions. In some embodiments the process of FIG. 15 performs operations of blocks 1411 and 1413 of the process of FIG. 14.

In block 1511 the process determines if the use a sleep profile with multiple beds is indicated as desired. In some embodiments use of the sleep profile with multiple beds is indicated as desired if an indication has been received indicating that the sleep profile may be used on multiple beds. In some embodiments the smartphone receives the indication, for example by way of a user input, which may be selection of an option allowing for use of the sleep profile on multiple beds. In some embodiments the smartphone provides the indication to the server. In some embodiments a compute device of a bed system receives the indication, for example by way of a user input, and in some embodiments the compute device may provide the indication to the server. In some embodiments, another compute device, for example a personal computer, may receive the indication, for example by way of a browser web page or application executing on the other compute device, and in some embodiments the other compute device may provide the indication to the server.

If use of the sleep profile on multiple bed is indicated as desired, the process continues to block 1513. Otherwise the process returns.

In block 1513 the process determines if the sleep profile should be stored on a personal compute device, a local mobile device in many embodiments, for example the smartphone of the user with which the sleep profile is associated, or in the “cloud”, for example on a server accessible via the Internet. In some embodiments the process determines the sleep profile should be stored on the smartphone if a privacy setting for the sleep profile indicates local storage only or private storage only. In some embodiments the process determines the sleep profile should be stored in a server accessible via the Internet if a user has selected, for example using an application on the smartphone, an option for storage of the sleep profile in the cloud.

If the process determines the sleep profile should be stored on the smartphone (or other user device), the process continues to block 1515a. If the process determines the sleep profile should be stored on a server accessible via the Internet, the process continues to block 1515b.

In block 1515a the process stores the sleep profile on a user device. The user device may be, for example, the smartphone. In some embodiments the bed transmits the sleep profile to the smartphone, for storage in memory of the smartphone.

In block 1515b the process stores the sleep profile on a server accessible via the Internet. In some embodiments the bed transmits the sleep profile to the server. In some embodiments the bed transmits the sleep profile to the smartphone, which in turn transmits the sleep profile to the server.

The process thereafter returns.

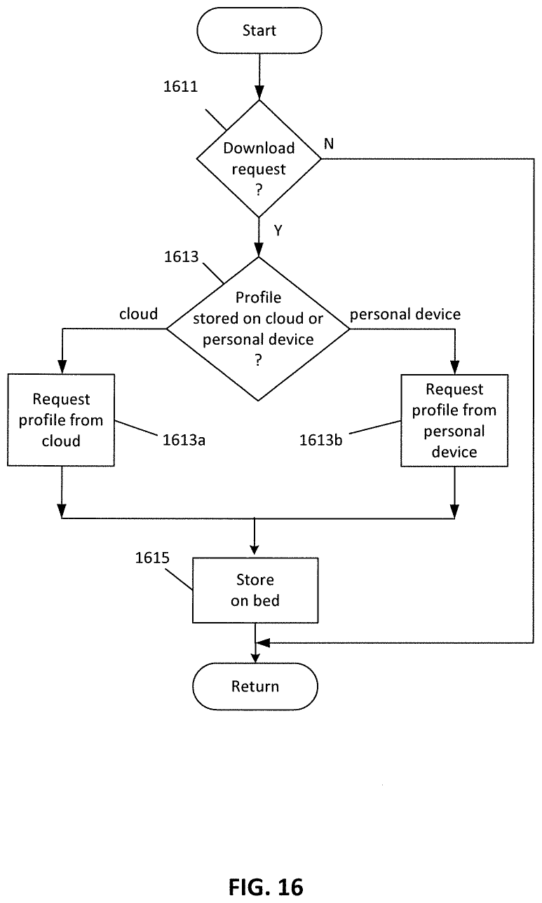

FIG. 16 is a flow diagram of a process for obtaining a sleep profile of a user for a bed, of a plurality of beds, in accordance with aspects of the invention. In some embodiments the process is performed by a system, for example the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a personal compute device, for example a smartphone and a controller of a bed with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions. In some embodiments the process performs operations of blocks 1415 and 1417 of the process of FIG. 14.

In block 1611 the process determines if a request has been made to download a sleep profile to a bed. In some embodiments the process determines that the request has been made if the process receives a request indicative of a request to download the sleep profile. In some embodiments the request is based on receipt of an input to a smartphone or compute device of a bed system indicating that the sleep profile should be downloaded to the bed. In some embodiments the request includes an identification of a user and/or a sleep profile associated with the user, and the bed.

If the process determines that the request has been made, the process continues to block 1613. Otherwise the process returns.

In block 1613 the process determines if the sleep profile is stored on a server accessible via the Internet or on a personal compute device. In some embodiments the request to download the sleep profile indicates whether the sleep profile is stored on the server or on the personal compute device. If the sleep profile is stored on the server, the process continues to block 1613a. If the sleep profile is stored on the personal compute device, the process continues to block 1613b. In some embodiments, for example embodiments in which the sleep profile is stored on the personal compute device and the request is based on a receipt of an input to the personal compute device, the process may forego operations of block 1613 and/or 1613b, with operations of later discussed block 1615 performed.

In block 1613a the process requests download of the sleep profile from the server. In some embodiments the request is made by the bed. In some embodiments the request is made by the personal compute device. In some embodiments the request is made by another compute device. In some embodiments the other compute device is, for example, a personal computer of the user, for example executing a web browser or other application. In some embodiments the other compute device is, for example, a computer or server of a reservation system. In some embodiments the request includes an identification of the bed to which the sleep profile should be downloaded. In some embodiments the request includes an identification of the user associated with the sleep profile. In some embodiments the request includes an identification of the sleep profile.

In block 1613b the process requests download of the sleep profile from the personal compute device. In some embodiments the request is made from the bed to the personal compute device. In some embodiments the request is made by a server. In some embodiments the server is a server of a reservation system. In some embodiments the request includes an identification of a bed to which the sleep profile should be downloaded.

In block 1615 the process downloads the sleep profile to the bed, and stores the sleep profile in memory of the bed. In some embodiments the sleep profile is transmitted by the smartphone to the bed. In some embodiments the server transmits the sleep profile to the bed.

The process thereafter returns.

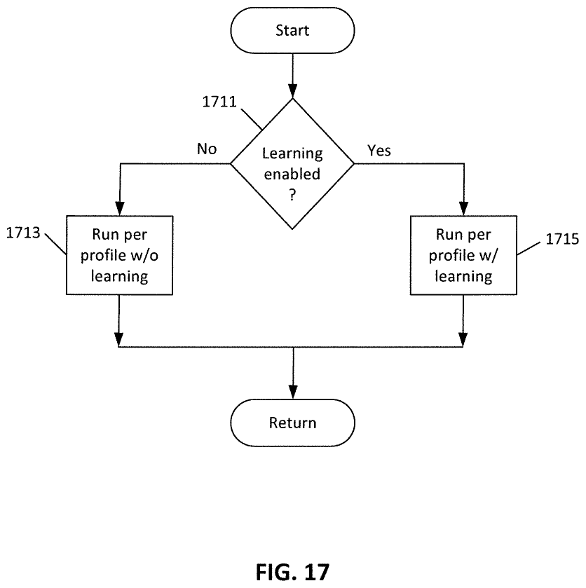

FIG. 17 is a flow diagram of a process for use in conditioning a bed, of a plurality of beds, for use by a user in accordance with aspects of the invention. In some embodiments the process is performed by a system, for example the system of FIG. 11. In some embodiments the process is performed by a server and a bed of the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a personal compute device, for example a smartphone, and a controller of a bed with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions. In some embodiments the process performs operations of blocks 1415 and 1417 of the process of FIG. 14.

In block 1711 the process determines if learning is allowed for use of a bed. In some embodiments the process determines that learning is allowed if the bed is a bed identified by a user as a bed normally used by the user. In some embodiments beds normally used by the user are considered to be beds which the user indicates the user owns, or is a bed of the user. In some embodiments the process determines that learning is allowed for all beds. In some embodiments the process determines that learning is allowed based on a selection by the user, for example a selection entered into a personal compute device of the user, a compute device associated with a bed, or some other compute device. In some embodiments the selection may indicate that learning is enabled for all beds, learning is enabled for one or more specific beds, or learning is enabled for no beds. In some embodiments the selection may indicate that the user does not have a sleep profile, but learning should be allowed for a particular bed, particular beds, or all beds, along with generation of a sleep profile for the user, in some embodiments.

If learning is not allowed for the bed, the process continues to block 1713. If learning is allowed for the bed, the process continues to block 1715.

In block 1713 the process conditions a sleep environment of the bed, based on information of the sleep profile. In some embodiments, in conditioning the sleep environment, a controller of the bed commands temperature adjustment components and/or pressure adjustment components of the bed in accordance with information of the sleep profile. In some embodiments the process performs operations as discussed with respect to block 613 of the process of FIG. 6, without performance of learning functions or operations.

In block 1715 the process also conditions the sleep environment of the bed, based on information of the sleep profile. In addition to operations discussed with respect to block 1713, however, the process additionally performs learning functions or operations, for example as discussed with respect to block 613 of the process of FIG. 6. As part of such learning operations, the process, in various embodiments, updates the sleep profile as a result of the learning operations. In some embodiments, the process also updates the sleep profile on the server, or, for example if the user does not allow the sleep profile to be stored on the server, updates the sleep profile on the user's smartphone.

The process thereafter returns.

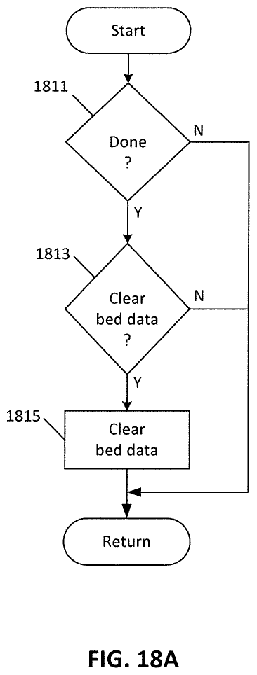

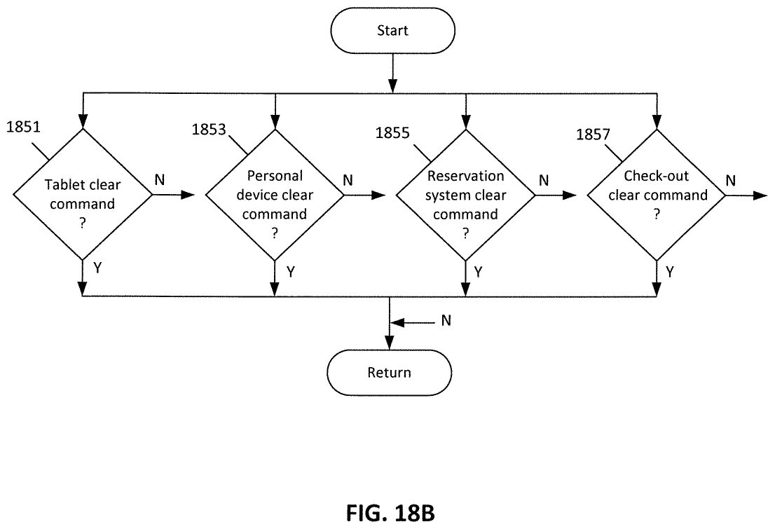

FIG. 18A is a flow diagram of a process for clearing sleep profile information of a user from a bed, in accordance with aspects of the invention. In some embodiments the process is performed by a system, for example the system of FIG. 11. In some embodiments the process is performed by a server and a bed of the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a personal compute device, for example a smartphone, and a controller of a bed with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions. In some embodiments the process performs operations of block 1421 of the process of FIG. 14.

In block 1811, which is optional in some embodiments, the process determines if use of the sleep profile on the bed is complete. In some embodiments the process determines that use of the sleep profile is complete if sensors of the bed indicate a user has left the bed and an expected wake time has passed. In some embodiments the process determines that use of the sleep profile is complete if information regarding the user indicates that the user will not be using the bed for a next sleep session. In some embodiments the information regarding the user is calendar information of the user, for example as stored on a smartphone of the user. In some embodiments the information regarding the user is information regarding an end date of a hotel reservation for the user, for example in circumstances in which the bed is in a guest room of the hotel.

If the process determines that use of the sleep profile on the bed is not complete, the process returns. Otherwise the process continues to block 1813.

In block 1813 the process determines if the sleep profile should be deleted from the bed. In some embodiments the process determines that the sleep profile should be deleted from the bed in response to receiving an input on a compute device of a bed system, for example the bed system of FIG. 13, with the input indicating a request to delete the sleep profile. In some embodiments the process determines that the sleep profile should be deleted from the bed in response to receiving an input on a smartphone of the user for the sleep profile, with the input indicating a request to delete the sleep profile from the bed. In some embodiments the process determines that the sleep profile should be deleted from the bed in response to receipt of a message from another computer, for example a server of a hotel reservation system, indicating that the user has checked out of a hotel room containing the bed.

If the process determines that the sleep profile should be deleted from the bed, the process continues to block 1815. Otherwise the process returns.

In block 1815 the process deletes the sleep profile from the bed. In some embodiments a controller of the bed commands deletion of the sleep profile from memory of the bed. In some embodiments the controller commands the deletion in response to receiving a message requesting deletion of the sleep profile. In some embodiments the message requesting deletion is from a compute device, for example a tablet computer, of a bed system including the controller. In some embodiments the message requesting deletion is from a smartphone of the user for the sleep profile. In some embodiments the message requesting deletion is from another computer, for example a computer of a hotel, with the computer linked by a local area network, or a local area network and the Internet, to the bed.

The process thereafter returns.

FIG. 18B is a flow diagram of a process for determining if a sleep profile on a bed should be cleared, in accordance with aspects of the invention. In some embodiments the process is performed by a system, for example the system of FIG. 11. In some embodiments the process is performed by a server and a bed of the system of FIG. 11. In some embodiments the process is performed, at least in part, by components of the system of FIG. 11, or the bed system of FIG. 13. In some embodiments the process is performed by a bed with a heating and/or cooling component and/or pressure adjustment component. In some embodiments the process, or parts of the process, is performed by a smartphone and a controller of a bed with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions. In some embodiments the process performs operations of block 1813 of the process of FIG. 18B.

The flow diagram of FIG. 18B indicates that a bed data should be cleared in the event of a variety of conditions. In some embodiments only one of the conditions is evaluated, in some embodiments only some of the conditions are evaluated, and in some embodiments all of the conditions are evaluated. In addition, although the flow diagram of FIG. 18B indicates parallel processing for evaluating conditions, in some embodiments conditions, or some of them, may be evaluated sequentially.

In block 1851 the process determines if a clear command has been received by a compute device associated with the bed. In some embodiments the compute device is, for example, a tablet computer, or other compute device with a display screen. In some embodiments the compute device displays a selectable option for commanding clearing of sleep profiles from the bed. In some embodiments the compute device includes a physical button for commanding clearing of sleep profiles from the bed. If the clear command has been received by the compute device, the process indicates that sleep profiles should be cleared from the bed.

In block 1853 the process determines if a clear command has been received by a personal compute device associated with a sleeper having a sleep profile stored on the bed. In some embodiments the compute device is a smartphone. In some embodiments the compute device displays a selectable option for commanding clearing of sleep profiles from the bed. In some embodiments the personal compute device provides a signal to a compute device associated with the bed indicating the sleep profiles should be cleared from the bed. In some embodiments the personal compute device provides a signal to a controller of the bed indicating the sleep profiles should be cleared from the bed. If the clear command has been received by the personal compute device, the process indicates that sleep profiles should be cleared from the bed.

In block 1855 the process determines if a clear command has been determined to be appropriate by a reservation system associated with the bed. In some embodiments the reservation system is configured to determine that the clear command is appropriate upon receipt of an input to a user interface of the reservation system requesting clearing of sleep profiles from a bed or beds in a room. In some embodiments the reservation system is configured to determine that the clear command is appropriate upon an end of a reservation period for a room including the bed. In some embodiments the reservation system is configured to determine that the clear command is appropriate upon receiving or determining that the sleeper has checked out of the room. If the clear command is determined to be appropriate, the process indicates that sleep profiles should be cleared from the bed.

In block 1857 the process determines if another computer indicates the sleeper is no longer using the room including the bed. In some embodiments the other computer is a computer of a hotel. In some embodiments the computer may be a computer executing a web browser, for example providing for interaction with a server, or application providing functions related to sleep profiles and their usage on beds. If the other computer indicates the sleeper is no longer using the room including the bed, the process indicates that sleep profiles should be cleared from the bed.

The process thereafter returns.

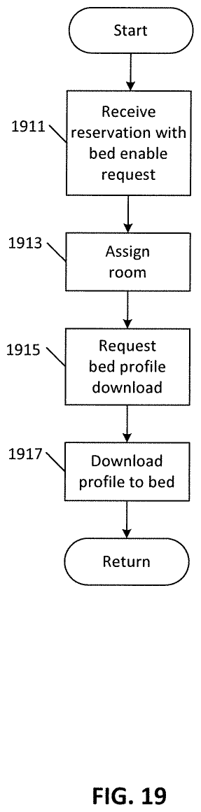

FIG. 19 is a flow diagram of a further process for providing a sleep profile to a bed, in accordance with aspects of the invention. In some embodiments the process performs operations of block 1417 of the process of FIG. 14. In some embodiments the process is for providing a sleep profile to a bed in a hotel or the like. In some embodiments the process is performed by a system, for example the system of FIG. 11. In some embodiments the process is performed, at least in part, by components of the system of FIG. 11, or the bed system of FIG. 13. In some embodiments the process is performed by at least one server and the bed system of FIG. 13. In some embodiments the at least one server is a server in a hotel reservation system. In some embodiments the process is performed by a bed with a heating and/or cooling component and/or pressure adjustment component. In some embodiments the process, or parts of the process, is performed by a personal compute device, for example a smartphone, and a controller or controllers of beds with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions.

In block 1911 the process receives information of a reservation for a room with an indication that a user for whom the reservation is made has a sleep profile to be used. The room, for example, includes a bed with pressure adjustment components and/or temperature adjustment components. The pressure adjustment components and/or temperature adjustment components may be controlled by a controller in accordance with information of at least one sleep profile. In some embodiments a server receives the information of the reservation for the room in the form of a completed request for a reservation of the room. In some embodiments the server is a part of a hotel reservation system. In some embodiments the indication that the user has a sleep profile to be used is an indication of the name of the user and/or a name of the sleep profile and/or an indication of a storage location, for example in the cloud, of the sleep profile.

In block 1913 the process assigns the room to the user. In some embodiments the room is assigned to the user during a check-process at the hotel. In some embodiments the room is assigned to the user by the reservation system. In some embodiments the bed to be used by the user is identified with assignment of the room to the user.

In block 1915 the process requests download of the sleep profile to the bed in the assigned room, or, if the room includes more than one bed, to the bed in the room to be used by the user. In some embodiments the reservation system sends information to the bed sufficient to allow for download of the sleep profile to the bed, and the bed requests the sleep profile from a server with access to the sleep profile. In some embodiments the reservation system requests the sleep profile from the server with access to the sleep profile. In some embodiments the reservation system may request the sleep profile from the server prior to the user being assigned the room.

In block 1917 the process downloads the sleep profile to the bed in the room assigned to the user, or to the bed assigned to the user in the room. In some embodiments the bed receives the sleep profile from the server in the cloud storing the sleep profile. In some embodiments the bed receives the sleep profile from the hotel reservation system.

The process thereafter returns.

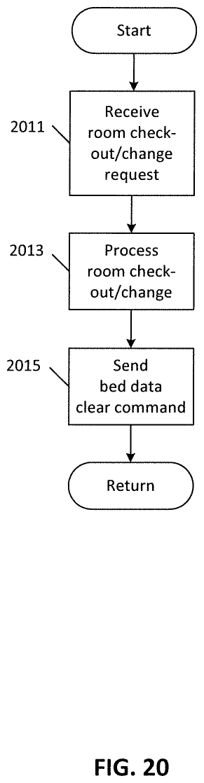

FIG. 20 is a flow diagram of a further process for clearing a sleep profile from a bed, in accordance with aspects of the invention. In some embodiments the process performs operations of block 1421 of the process of FIG. 14. In some embodiments the process is for providing a sleep profile to a bed in a hotel or the like. In some embodiments the process is performed by a system, for example the system of FIG. 11. In some embodiments the process is performed, at least in part, by components of the system of FIG. 11, or the bed system of FIG. 13. In some embodiments the process is performed by at least one server and the bed system of FIG. 13. In some embodiments the at least one server is a server in a hotel reservation system. In some embodiments the process is performed by a bed with a heating and/or cooling component and/or pressure adjustment component. In some embodiments the process, or parts of the process, is performed by a personal compute device, for example a smartphone, and a controller or controllers of beds with a heating and/or cooling component and/or pressure adjustment component. In some embodiments the process is performed, or parts of the process, by a compute device, for example at least one tablet computer, and a controller or controllers of beds with a heating and/or cooling component and/or pressure adjustment component. In some embodiments the process is performed by a controller, which may be a processor, for example configured by program instructions.

In block 2011 the process receives a room check-out or room change request. In some embodiments the hotel reservation system receives the room check-out or room change request.

In block 2013 the process processes the room check-out or room change request. In some embodiments the process processes the room check-out or room change request by storing an indication that the user no longer is checked into the room.

In block 2015 the process sends a request to the bed to delete the user sleep profile from the bed. In some embodiments the process sends the request as part of the processing of the room check-out or change request. In some embodiments the process sends the request in response to the indication that the user no longer is checked into the room being stored. In some embodiments the hotel reservation system transmits the request to the bed. In some embodiments the controller of the bed deletes the user sleep profile from the bed, in response to receiving the request.

The process thereafter returns.

Although the invention has been discussed with respect to various embodiments, it should be recognized that the invention comprises the novel and non-obvious claims supported by this disclosure.

What is claimed is:

1. A method for providing control of a sleep environment across multiple sleep platforms, comprising:
storing at least one sleep profile, for a user, on a server;
providing the at least one sleep profile to a first bed configured to condition a sleep environment based on one or more sleep profiles;
conditioning the sleep environment of the first bed using the at least one sleep profile;
providing information regarding the conditioned sleep environment of the first bed and sleep status of the user to the server for use in modifying the at least one sleep profile;
providing the at least one sleep profile to a second bed configured to condition a sleep environment based on one or more sleep profiles; and
conditioning the sleep environment of the second bed using the at least one sleep profile.

2. The method of claim 1, further comprising providing information regarding the conditioned sleep environment of the second bed and sleep status of the user to the server for use in modifying the at least one sleep profile.

3. The bed system of claim 1, wherein the at least one sleep profile is provided to the second bed in response to the user being assigned a room containing the second bed.

4. A method for providing control of a sleep environment across multiple sleep platforms, comprising:
receiving at least one sleep profile from a first bed configured to condition a sleep environment based on the at least one sleep profile;
transporting the at least one sleep profile to a second bed configured to condition a sleep environment based one or more sleep profiles; and
conditioning the sleep environment of the second bed using the at least one sleep profile.

5. The method of claim 4, wherein the at least one sleep profile is transported to the second bed using a smartphone.

6. The method of claim 5, wherein the smart phone receives the at least one sleep profile from the first bed by way of wireless communications, and the smart phone transmits the at least one sleep profile to the second bed by way of wireless communications.

7. The method of claim 4, wherein the at least one sleep profile is transported to the second bed by transmitting the at least one sleep profile over a network from the first bed to the second bed.

8. The method of claim 4, wherein the at least one sleep profile is stored on a server.

9. The method of claim 8, wherein the at least one sleep profile is transmitted from the first bed to the server over a network.

10. The method of claim 9, wherein the at least one sleep profile is transmitted from the server to the second bed over a network.

11. The method of claim 4, further comprising determining that the sleep profile should be deleted from the second bed; and deleting the sleep profile from the second bed in response to determining that the sleep profile should be deleted from the second bed.

12. The method of claim 11, wherein the determination that the sleep profile should be deleted from the second bed is made based upon a determination that a user associated with the sleep profile has checked out of a hotel.

13. The method of claim 12, wherein the determination is made based upon an input made into a compute device associated with the second bed.

14. A bed system, comprising:
a sleep surface;
sensors configured to sense sleepers on the sleep surface;
components for conditioning a sleep environment of the sleep surface;
a controller, within a frame of a bed of the bed system, configured to receive information from the sensors and to provide commands to the components for conditioning the sleep environment based on a sleep profile; and
a compute device, external to the frame of the bed of the bed system, configured to receive a request to delete the sleep profile from the bed system and configured to command deletion of the sleep profile from the bed system responsive to receipt of the request.

15. The bed system of claim 14, wherein the compute device comprises a tablet computer.

16. The bed system of claim 15, wherein the request to delete the sleep profile from the bed system comprises an input to the tablet computer indicating a request to delete the sleep profile from the bed system.

17. The bed system of claim 16, wherein the tablet computer is configured to transmit, to the controller, a command to delete the sleep profile from the bed system.

18. The bed system of claim 17, wherein the controller is configured to delete the sleep profile from memory associated with the controller.
